(19) United States
(12) Patent Application Publication
(10) Pub. No.: US 2011/0229523 A1
(43) Pub. Date: Sep. 22, 2011

(54) MODIFICATION OF ALLERGENS FOR IMMUNOTHERAPY

(75) Inventors: Stefan Johan Koppelman, Eg De Bilt (NL); Robertus Henricus Joannes Alfonsus Van Den Hout, Vd Haarlem (NL); Henriette Emilie Sleijster-Selis, Je Castricum (NL); Dionisius Marinus Antonious Maria Luijkx, JJ Wageningen (NL)

(73) Assignee: HAL Allergy Holding B.V., CH Leiden (NL)

(21) Appl. No.: 13/002,485

(22) PCT Filed: Jul. 6, 2009
(86) PCT No.: PCT/EP2009/058533

§ 371 Date: May 26, 2011

Related U.S. Application Data
(60) Provisional application No. 61/086,420, filed on Aug. 5, 2008.

(30) Foreign Application Priority Data
Jul. 4, 2008 (EP) 08159687.6

(51) Int. Cl. A61K 39/35; C07K 1/00; A61P 37/08; C07K 14/00

(52) U.S. Cl. 424/275.1; 530/403; 530/350

(57) ABSTRACT

The present invention relates to pharmaceutical compositions for immunotherapy, for example for immunotherapy of peanut allergy. Further, the present invention relates to methods for the preparation of the present pharmaceutical compositions for immunotherapy, and their use in immunotherapy.

Furthermore, the present invention relates to processes for modifying allergens thereby enhancing their application in immunotherapy. The invention also relates to the present modified allergens and pharmaceutical compositions comprising the present allergens, as well as to the use thereof in immunotherapy.

According to a particularly preferred embodiment, the present invention relates to pharmaceutical compositions for immunotherapy comprising reduced and alkylated naturally occurring peanut allergens Ara h2 and/or Ara h6, or derivates or isoforms thereof wherein said pharmaceutical composition substantially does not comprise Ara h1 and/or Ara h3.

The present invention relates to pharmaceutical compositions for immunotherapy, for example for immunotherapy of peanut allergy. Further, the present invention relates to methods for the preparation use of the present pharmaceutical compositions for immunotherapy. Furthermore, the present invention relates to processes for modifying allergens thereby enhancing their application in immunotherapy. The present invention also relates to the modified allergens and pharmaceutical compositions comprising the modified allergens, as well as to the use thereof in immunotherapy.

Allergens are substances that can cause an allergic reaction or induce an allergy. In individuals suffering from an allergy, allergens are recognized by the immune system as “foreign” or “dangerous”, whereas they cause substantially no response in most other people. Examples of common allergens are, or are present in, bacteria, viruses, animal parasites, insect venoms, house mites, chemicals, dust, medicaments such as antibiotics, foods, perfumes, plants, pollen, and smoke.

Food allergy is predominantly associated with a limited range of food products such as peanuts, tree nuts, hen's eggs, cow's milk, wheat (gluten), soybeans, fish and shellfish. The prevalence of food allergy is approximately 1 to 2% in adults and 6 to 8% in children.

The occurrence of allergic reactions is associated with a response of an individual's immune system to exposure of a particular allergen. In an individual susceptible for developing an allergy to a particular allergen, first time exposure to the allergen generally does not give rise to any allergic reactions.

Allergen are generally internalized by antigen presenting cells (APCs), such as macrophages or dendritic cells, which degrade, or digest, the allergen. Fragments of the allergen are presented to CD4+ T-cells, which may respond in essentially two different ways.

T-cells secrete cytokines which have effects on other cells of the immune system, most notably B-cells. They are subdivided into two categories. The first category contains T-helper1-cells, secreting amongst others interleukin-2 (IL-2) and interferon-γ (IFN-γ). The presence of IFN-γ will induce B-cells to produce specific subclasses of IgG antibodies.

The second category contains T-helper2-cells. These secrete different cytokines such as IL-4, IL-5 and IL-13. Production of IL-4 and IL-13 are necessary for the initiation, and maintenance, of IgE antibodies produced by B cells.

Upon additional exposure of the individual to a particular allergen, the allergen will bind to the available IgE antibodies and particularly to those bound to the surface of mast cells or basophils. As allergens typically have several sites that can bind to the IgE antibodies, those antibodies in effect become crosslinked. The result of the crosslinking of the surface-bound IgE antibodies is that the mast cells and basophils degranulate and release mediators like histamines that trigger allergic reactions.

Treatment of allergies is difficult. Many allergic individuals try to adapt to their situation and avoid exposure to the substances to which they are allergic. The feasibility of such behavior adaptation will depend of course to a great extent on the type of allergy. For instance, it is easier to avoid intake of certain foodstuffs than it is to avoid exposure to pollen.

Therapies involving drugs, such as antihistamines, decongestants, or steroids, are available but only combat the symptoms of an allergic reaction. They do not prevent that future exposure to the allergen causing new allergenic reactions.

It has been proposed to treat allergies on the basis of immunotherapy. Such treatment generally involves repeated injections of allergen extracts over a long period of time to desensitize a patient to the allergen. This therapy is, however, very time consuming, usually involving years of treatment, and frequently fails to achieve the goal of desensitizing the patient to the allergen.

Moreover, particularly for food allergies or allergies to insect venoms, it is not a safe treatment. With many food and insect venom allergies, allergic reactions are associated with a significant risk of anaphylaxis, which is a systemic and potentially lethal type of allergic reaction. In clinical trials for immunotherapy for peanut allergy, anaphylactic events occurred, once with fatal outcome.

In order to reduce the anaphylactic adverse events observed during insect venom immunotherapy, pre-treatment with antihistamines is recommended, indicating the poor safety profile of current immunotherapy for insect venoms.

It has been proposed to modify allergens to reduce the risk of such dangerous side effects. The modification aims to reduce the allergenic reactions caused by the allergen, while retaining its immunogenicity. Thus, ideally exposure to the modified allergen by an allergy patient would elicit the desired immune response so that, in time, the patient is desensitized to the allergen without causing severe allergic reactions during the therapy.

A known modification of protein allergens is a treatment with glutaraldehyde, which causes cross-linking of the allergen. The aldehyde groups of this glutaraldehyde react with the amino groups of lysine residues in the protein, or with the N-terminus. When the two aldehyde groups of glutaraldehyde react with amino groups of different proteins, a cross-link is made. For allergens, such cross-linking may lead to cross-linked material of variable size, with altered immunological characteristics.

For some allergens, like from tree pollen or grass pollen, it has been demonstrated that modification with glutaraldehyde results in a reduced IgE-binding, which, in turn, reduces adverse side effects of immunotherapy. It is believed that lysine residues in allergens may be involved in IgE-epitopes, and that modification or cross-linking of these lysine residues leads to diminished IgE binding due to alterations in the conformation of the protein structure.

However, the ability of glutaraldehyde-treated allergens to stimulate T-cells has been disputed. Furthermore, it has been found that treatment with glutaraldehyde is not always suitable. In particular, the IgE binding of various allergens, such as peanut allergens, modified by treatment with glutaraldehyde is not reduced.

In WO 2005/060994, it has been disclosed that food allergens, in particular seed storage proteins such as 2S albumins, may be modified by reduction and alkylation. It is postulated that this treatment results in both breakage of disulfide bonds and prevention of reformation of disulfide bonds.

The modification is stated to result in a reduction or even prevention of the production specific IgE antibodies after presenting an individual's immune system with the modified allergen. However, IgE-binding itself was not investigated.

Furthermore, it has been found that the allergenicity of the allergens in some cases needs to be reduced even further, without reducing immunogenicity, for them to be suitable and safe in an effective immunotherapy.

For instance, some allergens, such as wasp or bee venom, tend to provoke notoriously severe allergic reactions so that immunotherapy for this type of allergies has hitherto not been sufficiently safe. Furthermore, the allergens modified in accordance with this prior art document are not always sufficiently stable as reduction of disulfide bridges of highly structured proteins leads to increased susceptibility for proteolysis and heat denaturation.

The present invention provides an improved way of modifying allergens which greatly reduces the allergenicity of allergens, essentially without detrimentally affecting their immunogenicity. A modification according to the invention can be used for a great variety of allergens.

Because the allergens modified according to the invention display a significantly improved safety profile compared to currently available allergen products, the invention provides a means to improve existing immunotherapies.

In particular, allergy patients will experience no, or less severe, adverse side effects of the immunotherapy when using allergens modified according to the invention. Also, the efficacy of the treatment may be improved as it will be possible to treat patients with higher dosages of allergens which, in turn, may decrease the time for the patient to become tolerant. In addition, the invention provides a means to develop immunotherapy for allergies that are caused by allergens which are unsuitable for immunotherapy since their allergenicity cannot be sufficiently modified with currently available methods.

Peanut allergy is both common and frequently severe. Peanut allergens have been characterized to a great extent over the last decade, and various purification protocols have been published for some of the allergens.

A major peanut allergen, designated as Ara h1, see for example GI: 193850561, was described as a 63.5 kDa protein occurring naturally in a trimeric form of approximately 180 kDa through non-covalent interactions. The trimeric Ara h1 structures often aggregate, forming multimers of up to 600-700 kDa.

The second identified major peanut allergen Ara h2, see for example GI: 26245447, migrates as a doublet at approximately 20 kDa. This doublet consists of two isoforms that are nearly identical except for the insertion of the sequence DPYSPS in the higher molecular weight isoform.

Ara h3, see for example GI: 112380623, is a more complex allergen. After its initial identification as a 14 kDa protein, a full gene encoding a 60 kDa protein was successfully expressed. Purification of Ara h3 showed that in the peanut kernel Ara h3 is present as a post-translational and proteolytically processed protein consisting of a triplet at approximately 42 to 45 kDa, a distinct band at approximately 25 kDa, and some less abundant peptide chains in the 12 to 18 kDa range.

Another peanut allergen, designated as Ara h6, see for example GI: 148613182 or GI:148613179, was identified as a protein with a molecular weight of approximately 15 kDa based on SDS-PAGE and 14,981 Da as determined by mass spectroscopy.

Several other peanut allergens, designated as Ara h4, h5, h7, h8, and h9, have also been described.

Considering the potential severity of peanut allergies, in some cases even fatal, it is an object of the present invention, amongst other objects, to provide pharmaceutical compositions, and methods for their preparation, suitable for immunotherapy of peanut allergies as well as the application thereof for immunotherapy of peanut allergies.

This object, amongst other objects, is met according to the present invention by a pharmaceutical composition as defined in the appended claim 1.

Specifically, this object is met according to the present invention by a pharmaceutical composition for immunotherapy comprising:
reduced and alkylated naturally occurring Ara h2 and/or Ara h6, or and derivates or isoforms thereof;
   wherein said pharmaceutical composition substantially does not comprise Ara h1 and/or Ara h3.

According to the present invention, a suitable isoform of Ara h2 is Ara h7.

The present inventors surprisingly recognized that by exposing peanut allergy patients to the present allergens Ara h2 or Ara h6, or, and preferably, a combination thereof, patients can be effectively treated using immunotherapy.

Although reduced IgE binding can be recognized as one of the factors contributing to the effectivity of the present immunotherapy, it should be realized that reduced IgE binding is only one of the many factors to be considered for an effective immunotherapy.

The route and/or way of presentation of an antigen, such as Ara h2 and/or Ara h6, to the immune system influence available epitopes. The presentation is, amongst others, depending on the stability and/or digestibility of an allergen. For example, ingested allergens digested in the stomach will be differently presented than less digested allergens. In general, less digested allergens will provide a larger repertoire of immunogenic epitopes than digested allergens which are mainly presented to the immune system as smaller fragments or peptides inherently comprising a smaller repertoire of epitopes.

For an effective immunotherapy, a careful balance must be found between desensitizing an immune response and the induction of a strong, potentially dangerous, allergenic reaction. Binding of an antigen to its complementary receptors on a T or B lymphocyte can stimulate the lymphocyte to divide and mature, thereby providing the initiation of a potential allergic reaction, or the binding can eliminate, or inactivate, the lymphocyte, thereby providing immune tolerance or immunotherapy.

For such as balance, the specific choice of allergens is of critical importance. The allergens must carry the potential to interact with the immune system but, on the other hand, the immune system must not be stimulated to cause an allergic response.

Although it is known that reducing IgE responses contributes to a decreased immunogenicity of an allergen, thereby shifting the balance towards tolerance, also other important factors to be considered are, for example, the maturity of the lymphocyte, the nature and concentration of the antigen and complex interactions between different classes of lymphocytes and between lymphocytes and antigen-presenting cells, all contributing to either an immune response or tolerance.

The present inventors surprisingly recognized that the above balance between immune response and tolerance could be found in Ara h2 or Ara h6, or, and preferably, a combination thereof, modified and substantially without the presence of other peanut allergens such as Ara h1 and/or Ara h3.

Considering the delicate balance between immune tolerance and response, the present inventors recognized that it is of critical importance that the present allergens Ara h2 and/or Ara h6, and isoforms or derivatives thereof, are as closely as possible identical to the antigens present in peanut.

Accordingly, the present invention according to this aspect solely resides in naturally occurring antigens and not, for example, artificially produced antigens such as in a recombinant expression system. Inherently, the use of recombinant allergens will introduce deviations, such as post-translational processing and glycosylation, from the natural occurring allergens thereby affecting, or disturbing, the present balance towards immune tolerance providing an effective immunotherapy.

Accordingly, the present allergens are naturally occurring allergens, i.e., derived, isolated, or originating, from a natural source such as peanuts or processed forms thereof.

According to a preferred embodiment, the present invention relates to pharmaceutical compositions additionally comprising one or more adjuvants, preferably comprising Aluminium, and/or pharmaceutically acceptable excipients and/or carriers.

According to another preferred embodiment, the present invention relates to pharmaceutical compositions wherein the present reduced and alkylated Ara h2 and/or Ara h6 are additionally crosslinked.

Considering the through the present pharmaceutical compositions provided beneficial immune tolerance, or immunotherapy, the present invention, according to another aspect, relates to a method for preparing a pharmaceutical composition for immunotherapy comprising:
providing a composition comprising naturally occurring Ara h2 and/or Ara h6, or isoforms or derivatives thereof, wherein said composition substantially does not comprise Ara h1 and/or Ara h3;
reducing said composition; and
alkylating the reduced composition.

According to a preferred embodiment of this aspect, providing according to the present method comprises purifying Ara h2 and/or Ara h6, or isoforms or derivatives thereof.

According to yet another preferred embodiment of this aspect, the present method further comprises crosslinking the present reduced and alkylated composition.

According to more preferred embodiments, the present method further comprises formulating the reduced and alkylated composition with one or more adjuvants, preferably Aluminium, and/or pharmaceutically acceptable excipients or carriers.

Preferably, the present reducing comprises contacting the present composition with one or more reducing agents chosen from the group consisting of 2-mercaptoethanol (β-ME), dithiothreitol (DTT), dithioerythritol, cysteine, homocystein, tributylphosphine, sulfite, tris(2-carboxyethyl) phosphine (TCEP), sodium (cyano) borohydride, lye, glutathione, E-mercapto ethylamine, thioglycollic acid, methyl sulfide, and ethyl sulfide.

Preferably, the present alkylating comprises contacting the present reduced composition with one or more alkylating agents chosen from the group consisting of N-ethylmalimide, cystamine, iodoacetamide, iodoacetic acid, alkylhalogenides; alkylsulfates; alkenes, preferably terminal alkenes (H₂C)═C(H)—R, and enzymes.

Preferably, the present crosslinking comprises contacting the reduced and alkylated composition with an aldehyde, preferably glutaraldehyde.

According to yet another aspect, the present invention relates to the use of a composition comprising naturally occurring Ara h2 and/or Ara h6, or isoforms or derivatives thereof, a pharmaceutical composition as defined above, or a pharmaceutical composition obtainable by the present methods for immunotherapy, i.e., inducing immune tolerance for peanuts thereby alleviating, or obviating, peanut allergy.

According to still another aspect, the present invention relates to a process for modifying an allergen comprising the steps of reduction and treatment with a cross-linking agent.

A modification according to this aspect of the present invention comprises the steps of reduction and treatment with a cross-linking agent, such as glutaraldehyde. Optionally, in a preferred embodiment, a modification according to the invention further comprises alkylation.

These steps may be carried out in any order, but it is preferred that reduction is carried out prior to alkylation, if it is included. Treatment with the cross-linking agent is preferably carried out after reduction and alkylation.

Surprisingly, exposure of an allergic individual to an allergen modified according to this aspect of the invention is not only safe and does alleviate or inhibit any significant allergic reactions, it is also possible to effectively desensitize the individual to the allergen.

Presenting the individual's immune system with an allergen modified in accordance with this aspect of the invention has been found to lead to a reduction or prevention of the production of specific-IgE antibodies. In an individual with a developed allergy, the IgE response of the immune system may be down-regulated skewing the immune response from a T-helper-2 mediated reaction towards a T-helper-1 mediated reaction, thereby reducing or alleviating the allergic reaction.

It is further advantageous that an allergen that is modified in accordance with this aspect of the invention is highly stable and very safe. Immunotherapy for allergies to highly dangerous allergens, such as, but not limited to, peanut or wasp or bee venom, has been made possible with allergens modified according to the invention.

The term “allergen” or “antigen” is used herein to refer to an agent which, when exposed to a mammal, will be capable of eliciting an immune response resulting, amongst others, in antibodies of the IgE-class and which also will be able to initiate or trigger an allergic reaction. Allergens in terms of the present invention are allergenic proteins, which may consist of protein or a protein combined with a lipid or a carbohydrate such as a glycoprotein, a proteoglucan, a lipoprotein etc.

In accordance with this aspect of the invention, the allergen typically is a protein, preferably a protein comprising cystein residues. More preferably, the allergen comprises cystein residues that form disulfide bridges or disulfide bonds, preferably intramolecular disulfide bonds. In the context of this aspect of the present invention, the terms “disulfide bridges” and “disulfide bonds” will be used interchangeably. It is further preferred, in the context of this aspect of the present invention, that the allergen is from a vegetable source, preferably a storage protein, from an insect, a mammal or a fish or crustacean, or from an expression system for recombinant proteins like a bacterium yeast or other microorganism.

Allergens from plants according to this aspect may be subdivided in allergens from pollen and the like and allergens from seeds. Allergens from seeds are preferably storage proteins such as 2S-albumin or conglutin. In purified form such storage proteins are, in a preferred embodiment, for instance Ara h2 and/or Ara h6 from peanut.

Alternatively, allergens from plants may be subdivided in allergens from fruit, such as lipid transfer proteins, allergens from oil crops, such as peanut or soybean, and allergens from treenuts and seeds such as hazelnut, walnut and sunflower seed. Allergens from insects are preferably venoms from for instance bee or wasp, which may be purified to obtain individual allergens.

Prior to modification, in the context of this aspect of the present invention, the allergen is preferably isolated (purified) from its biological source, such as (a part of) the animal, insect venom, foodstuff, or the like. It is, however, also possible to modify a crude, or partially purified extract comprising the allergen together with other components of the biological source. Although this may result in administration to a patient of other proteins or other substances modified by reduction and treatment with a cross-linking agent, this is not considered to be harmful.

Therefore, according to another aspect, the present invention pertains to modification of isolated allergens as well as to crude extracts from allergen-containing products, such as food items, as obtainable by e.g. milling, grinding, etc. which have been subjected to modification according to the present invention. It is also possible to use mixtures of allergens, particularly mixtures of allergens from one source.

If desired, isolation of the allergen may be provided by any known method, such as methods involving extraction and liquid chromatography. Methods for isolating allergens from various biological sources are known per se and may be conveniently adapted to the needs of the circumstances by the skilled person based on his common general knowledge.

The allergen may also be obtained commercially, such as for instance from Greer, Lenoir, N.C., USA, from Indoor Biotech, Charlottesville, N.C., USA, from Allergon AB, Ängelholm, Sweden, from ALK Albello, Horsholn, Denmark, or from Pharmacia Diagnostics AB, Uppsala, Sweden.

It is further possible, according this aspect, to use allergens that have been obtained by recombinant means or to use synthetic peptides as allergen. Recombinant allergens are commercially available from for instance BioMay, Vienna, Austria. Synthetic peptides that can be used as allergens are commercially available from for instance Circassia, Oxford, UK.

In accordance with this aspect of the invention, the allergen is modified by reduction and treatment with a cross-linking agent. Preferably, the modification further comprises alkylation. As mentioned above, these three steps may be performed in any order, but it is preferred that treatment with the cross-linking agent is carried out after reduction and alkylation.

It is further preferred that reduction is carried out prior to alkylation. In another preferred embodiment, the allergen is modified by reduction, followed by treatment with the cross-linking agent, and finally by alkylation. In yet another embodiment, alkylation and reduction are carried out simultaneously by making use of a reagent that is capable both of reducing and alkylating proteins.

Performic acid may be used to oxidize disulfide bridges to sulfonates, thereby preventing re-oxidation. The reaction conditions should be chosen such that oxidation of methionine and tryptophane is avoided. Sulfite can be used to modify disulfide bridges into SO₃⁻ groups, thereby preventing re-oxidation in a similar way as 4,5-dihydroxy-1,2-dithiane and 2-({4-[(carbamoylmethyl)sulfanyl]-2,3-dihydroxybutyl}sulfanyl)acetamide do. In general, reductive alkylation in a single step may be applied to reduce disulfide bridges irreversibly in a single step.

Reduction and alkylation of proteins are protein modifications that are known per se. It will be understood that it is preferred that only reagents are used which lead to modified allergens that are acceptable in the context of the production of foodstuffs or pharmaceuticals.

In a preferred embodiment of the present invention, reduction is performed using a reducing agent chosen from the group of 2-mercaptoethanol (β-ME), dithiothreitol (DTT), dithioerythritol, cysteine, homocystein, tributylphosphine, sulfite, tris(2-carboxyethyl) phosphine (TCEP), sodium (cyano) borohydride, lye, glutathione, E-mercapto ethylamine, thioglycollic acid, methyl sulfide, ethyl sulfide and combinations thereof. In general, alkylthiol compounds (R—SH) provide suitable reducing agents. Preferably, those reducing agents are used that disrupt the disulfide bonds while maintaining other chemical characteristics of the protein. For instance, NH₂ groups are preferably left intact.

Alternatively, reduction according to the present invention may be performed by using enzymatic means, such as by using proteins that catalyse thiol-disulfide exchange reactions such as for instance glutaredoxin or thioredoxin. Such proteins may exert their effect via two vicinal (CXYC) cysteine residues, which either form a disulfide (oxidized form) or a dithiol (reduced form). Alternatively proteins may be used that are capable of catalysing the rearrangement of both intrachain and interchain-S—S-bonds in proteins such as protein disulfide isomerase or other polypeptides capable of reducing disulfide.

Preferably, the reduction reaction according to the present invention is continued until the reaction stops and essentially all disulfide bonds in the allergen have been broken. The conditions under which reduction is carried out can be optimized depending on the chosen reducing agent by the skilled person based on his general knowledge. Typically, reduction will be carried out at neutral, or near neutral pH, preferably at a pH between 6 and 8, at concentrations of reducing agents in the suitable range of, or equivalent to, for instance about 1-100 mM of DTT (or (3-ME), possibly by using a suitable buffer. An example of a suitable buffer comprises chaotropic reagents, such as guanidine and/or urea, which may result in (reversible) unfolding of the allergen protein. If such reagents are used, it is preferred that reduction and alkylation are performed before treatment with the cross-linking agent.

The temperature during reduction will generally lie between ambient or room temperature and 100° C., optionally under a reducing atmosphere, such as an anoxic atmosphere, preferably a nitrogen (N₂) atmosphere. Of course, care should be taken that the allergen does not denature during the reaction.

In a preferred embodiment, a modification according to this aspect of the invention not only comprises reduction and treatment with a cross-linking agent, but also alkylation.

Alkylation according to the present invention is preferably carried out by blocking the SH-radicals that result from the cleavage of the disulfide bonds during reduction. Preferred alkylation reagents are chosen from the group of N-ethylmaleimide, cystamine, iodoacetamide, iodoacetic acid.

More generally, at least one disulfide bond can be reduced and alkylated to produce cysteine residues with side chains having the chemical formula —CH₂—S—[CH₂]ₙ—R′ wherein n is an integer between 1 and 5 and R′ is selected from the 1-5 carbon groups consisting of alkyl groups (e.g., methyl, ethyl, n-propyl, etc.); carboxy alkyl groups (e.g., carboxymethyl, carboxyethyl, etc.); cyano alkyl groups (e.g., cyanomethyl, cyanoethyl, etc.); alkoxycarbonyl alkyl groups (e.g., ethoxycarbonylmethyl, ethoxycarbonylethyl, etc.); carbomoylalkyl groups (e.g., carbamoylmethyl, etc.); and alkylamine groups (e.g., methylamine, ethylamine, etc.). Other suitable alkylating reagents include alkylhalogenides; alkylsulfates; alkenes, preferably terminal alkenes (H₂C)═C(H)—R; and other alkylating reagents known to one skilled in the art.

Alternatively, alkylation according to the present invention may be performed by using enzymatic means, such as by using sulfhydryl oxidase, for instance as may be obtained from chicken egg protein. Although strictly not an alkylation reaction, the oxidation of the SH-radicals towards for instance SO₂ or SO₃ forms an aspect of the present invention since the reformation of the protein disulfide bonds is effectively blocked as a result thereof.

In some preferred embodiments of this aspect of the invention, the alkylation will introduce amino groups that may react with the cross-linking agent in embodiments where this step is performed after alkylation. This may be used as a further instrument to achieve a desired degree of modification of the allergen. Examples of suitable alkylation reagents in accordance with this embodiment are cystamine, iodoacetamide, acrylamide, and 2-({4-[(carbamoylmethyl)sulfanyl]-2,3-dihydroxybutyl}sulfanyl)acetamide.

Typically, alkylation according to the present invention will be carried out at neutral, or near neutral pH, preferably at a pH between 6 and 8, possibly be using a suitable buffer. An example of a suitable buffer comprises chaotropic reagents, such as guanidine and/or urea, that may result in unfolding of the allergen protein. If such reagents are used, it is preferred that reduction and alkylation are performed before treatment with the cross-linking agent. The temperature during alkylation will generally lie between ambient or room temperature and 50° C.

The allergen is, in accordance with this aspect of the invention, also treated with a cross-linking reagent. The cross-linking agent may be a bifunctional reagent, which may be a homo-bifunctional reagent or a hetero-bifunctional reagent. This means that it may comprise either two of the same functional moieties or that it may comprise two different functional moieties. By virtue of its bifunctionality, the bifunctional reagent may act as a cross-linking agent. However, other cross-linking agents, such as certain monoaldehydes, may also be used. The functional moieties of the cross-linking agent may react with certain amino acids in the allergen protein. For instance, aldehyde groups of a cross-linking moiety may react with the amino groups of lysine residues in the protein, or of the N-terminus In the case of formaldehyde, for instance, the product of this reaction is very reactive as a result of which both inter- and intramolecular cross-links may be formed.

Suitable examples of cross-linking agents are aldehydes, such as formaldehyde and glutaraldehyde. Preferably, the cross-linking agent is glutaraldehyde.

The crosslinking treatment according to the present invention may be performed at conditions that can be easily optimized by the skilled person based on his common general knowledge. It may comprise reacting the allergen with the cross-linking agent in a molar ratio of 10-100:1 of cross-linking agent to lysine residues, at highly alkaline pH, at room temperature for a few hours. The reaction may be stopped in any suitable way, for instance by addition of an excess of glycine followed by diafiltration.

In an alternative embodiment of this aspect, a process according to the invention comprises carbamylation of an allergen in addition to, or instead of, a treatment with a cross-linking agent. Carbamylation generally comprises treatment of the allergen with an alkaline cyanate, such as potassium cyanate, or with an organic isocynate, such as methyl isocyanate or methyl isothiocyanate, preferably in an alkaline environment, e.g. a pH between 9 and 9.6, and a temperature between ambient temperature and 50° C. This treatment will generally last between 12 and 36 hours.

The present invention also encompasses, according to yet another aspect, a modified allergen that can be obtained by the above described modification reactions. It is contemplated that an allergen modified as described above is produced directly by recombinant means at least according this aspect, or by means of peptide synthesis, without requiring the chemical modification steps as described herein.

It is further contemplated that an allergen partially modified as described above according to this aspect is produced directly by recombinant means or by means of peptide synthesis and that the remaining required modification steps are performed chemically as described herein. All of these (partially) recombinant modified and (partially) synthesized modified allergens are also encompassed by this aspect of the invention.

It will be understood that the invention also relates to a pharmaceutical composition comprising the modified allergen of this aspect for immunotherapy directed against allergy.

A pharmaceutical composition according to this aspect of the invention comprises a therapeutically effective amount of the polypeptides modified as described above.

Once formulated, the pharmaceutical compositions of the invention can be administered directly to the subject. Direct delivery of the compositions will generally be accomplished by injection, but the compositions may also be administered orally, nasally, rectally, mucosally, through the skin, subcutaneously, sublingually, intraperitoneally, intravenously, intralymphatically or intramuscularly, pulmonarily, or delivered to the interstitial space of a tissue.

The pharmaceutical composition according to the present invention may also comprise a suitable pharmaceutically acceptable carrier and may be in the form of a capsule, tablet, lozenge, dragee, pill, droplets, suppository, powder, spray, vaccine, ointment, paste, cream, inhalant, patch, aerosol, and the like.

As pharmaceutically acceptable carrier, any solvent, diluent or other liquid vehicle, dispersion or suspension aid, surface active agent, isotonic agent, thickening or emulsifying agent, preservative, encapsulating agent, solid binder or lubricant can be used which is most suited for a particular dosage form and which is compatible with the modified allergen.

It may be preferred to further include an adjuvant, preferably one known to skew the immune response towards a Thelper-1 mediated response, in the dosage form, in order to further stimulate or invoke a reaction of the patient's immune system upon administration.

Suitable adjuvants include such adjuvants as complete and incomplete Freund's adjuvant and aluminium hydroxide, the latter of which works through a depot effect.

It is also conceived that the modified allergen is incorporated in a foodstuff and is administered to a patient together with food intake.

A pharmaceutical composition according to the present invention may also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which may be administered without undue toxicity. Suitable carriers may be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art.

Pharmaceutically acceptable salts can be used therein, for example, mineral acid salts such as hydrochlorides, hydrobromides, phosphates, sulfates, and the like; and the salts of organic acids such as acetates, propionates, malonates, benzoates, and the like.

Pharmaceutically acceptable carriers in therapeutic compositions may contain liquids such as water, saline, glycerol and ethanol. Additionally, auxiliary substances, such as wetting or emulsifying agents, pH buffering substances, and the like, may be present in such vehicles. Typically, the therapeutic compositions are prepared as injectables, either as liquid solutions or suspensions; solid forms suitable for solution in, or suspension in, liquid vehicles prior to injection may also be prepared. Liposomes are included within the definition of a pharmaceutically acceptable carrier.

For therapeutic treatment, modified allergenic proteins may be produced as described above and applied to the subject in need thereof. The modified allergenic proteins, such as Ara h2 and/or Ara h6, may be administered to a subject by any suitable route, preferably in the form of a pharmaceutical composition adapted to such a route and in a dosage which is effective for the intended treatment.

Therapeutically effective dosages of the modified allergenic proteins required for decreasing the allergenic reaction to the native form of the protein or for desensitising the subject can easily be determined by the skilled person, e.g. based on the clinical guidelines for immunotherapy for allergy treatment. In particular, this is practiced for insect venoms.

The term “therapeutically effective amount” as used herein refers to an amount of a therapeutic, viz. a modified allergenic protein according to the present invention, to reduce or prevent allergic reactions to allergenic proteins, or to exhibit a detectable therapeutic or preventative effect.

The precise effective amount for a subject will depend upon the subject's size and health, the nature and extent of the condition, and the therapeutics or combination of therapeutics selected for administration. In case a subject has undergone treatment with antihistamines, dosages will typically tend to be higher than without such pre-treatment. Thus, it is not useful to specify an exact effective amount in advance. However, the effective amount for a given situation can be determined by routine experimentation and is within the routine judgment of the clinician or experimenter.

Specifically, the compositions of the present invention can be used to reduce or prevent allergic reactions to allergenic proteins and/or accompanying biological or physical manifestations. Such manifestations may include contraction of smooth muscle in the airways or the intestines, the dilation of small blood vessels and the increase in their permeability to water and plasma proteins, the secretion of thick sticky mucus, and, in the skin, redness, swelling and the stimulation of nerve endings that results in itching or pain.

Manifestations that may be prevented by immunotherapy according to the present invention include skin manifestations such as rashes, hives or eczema; gastrointestinal manifestations including cramping, nausea, vomiting or diarrhoea; or respiratory manifestations including sneezing or runny nose, coughing, wheezing or shortness of breath.

Other manifestations that may be prevented include itching of skin, flushes, congestion, eye irritation, asthma, itching in the mouth or throat which may progress to swelling and anaphylaxis. Methods that permit the clinician to establish initial immunotherapy dosages are known in the art (e.g. U.S. Pat. No. 4,243,651). The dosages to be administered must be safe and efficacious. As with any medical treatment, a balance must be struck between efficacy and toxicity.

For purposes of the present invention, an effective dose will be from about 0.1 ng/kg to 0.1 mg/kg, 10 ng/kg to about 10 μg/kg, or 0.1 μg/kg to 1 μg/kg of the modified allergenic protein relative to the body weight of the individual to which it is administered.

Often, a treatment will comprise starting with the administration of dosages at the lower end of these ranges and increasing the dosages as the treatment progresses. These dosages are intended for modified allergens obtained from purified allergens. For modified allergens based on a crude extract of the allergen, dosages may be higher corresponding to the purity of the extract used.

For typical desensitization treatment, it is generally necessary for the patient to receive frequent administrations, e.g., initially every two or three days, gradually reducing to once every two or three weeks. Other suitable desensitisation programs include subcutaneous injections once every 2-4 weeks the dosage of which injections may gradually increase over a period of 3-6 months, and then continuing every 2-4 weeks for a period of up to about 5 years. It is also possible, particular for sublingual administration, that daily administrations are given.

Desensitization protocols may also comprise a form of treatment conventionally known in various equivalent alternative forms as rapid desensitization, rapid allergen immunotherapy, rapid allergen vaccination, and rapid or rush immunotherapy. In broad terms, this procedure aims to advance an allergic patient to an immunizing or maintenance dose of extract (i.e., allergen) by administering a series of injections (or via another suitable carrier) of increasing doses of the allergen at frequent (e.g. hourly) intervals. If successful, the patient will exhibit an improved resistance to the allergen, possibly even presenting a total non-reactivity to any subsequent allergen exposure.

Various desensitization protocols are known in the art and may for instance comprise a method of treating a patient having an immediate hypersensitivity to an allergen using an accelerated rapid immunotherapy schedule in combination with a method of pre-treating such patient with prednisone and histamine antagonists prior to receiving the accelerated immunotherapy.

Yet in another alternative embodiment, the modified allergens or compositions of the invention may be administered from a controlled or sustained release matrix inserted in the body of the subject.

It will be understood, that the optimal dose and administration scheme of how to reach this dose may vary per patient. Based on his common general knowledge and taking due account of IgE-mediated side effects, the skilled person will be able to optimize dosage and administration scheme.

For example, in a 3 months period the allergen may be given weekly, with weekly increasing doses until a maintenance dose, e.g. 100 micrograms, is reached.

However, if treatment with this maintenance dose does not result in sufficient protection, the dose may be increased. An advantage of an allergen modified according to the invention is that it binds to IgE to a lower extent. This may prevent IgE-mediated side effects and allow quicker up-dosing.

The invention will further be detailed in the following examples of preferred embodiments of the present invention. In the examples, reference is made to figures wherein:

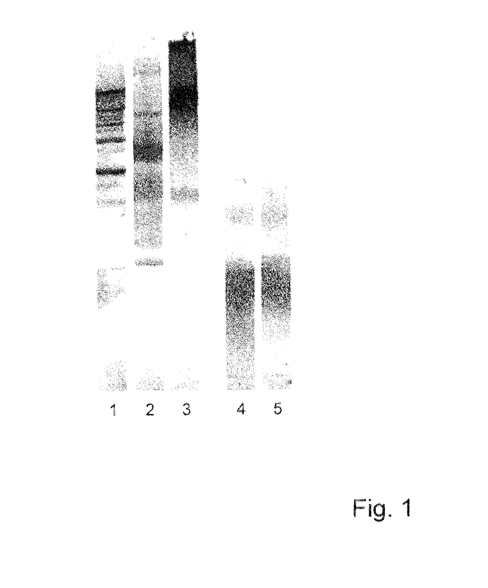

FIG. 1: shows an IgE-blot of glutaraldehyde treatment purified Ara h 1 and Ara h 2, showing marker proteins (lane 1), unmodified Ara h 1 (lane 2), modified Ara h 1 (lane 3), unmodified Ara h 2 (lane 4), modified Ara h 2 (lane 5).

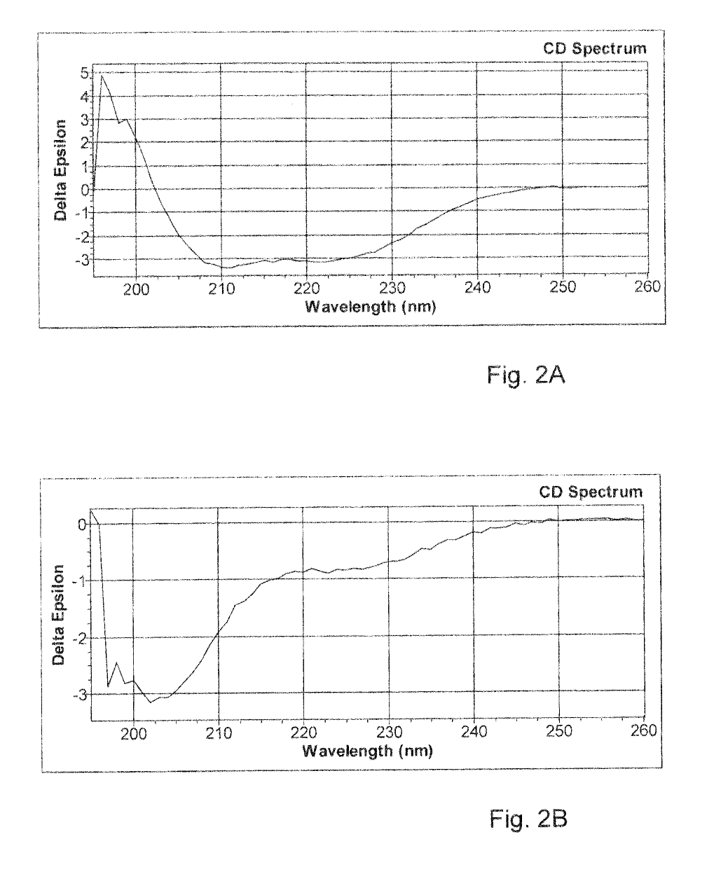

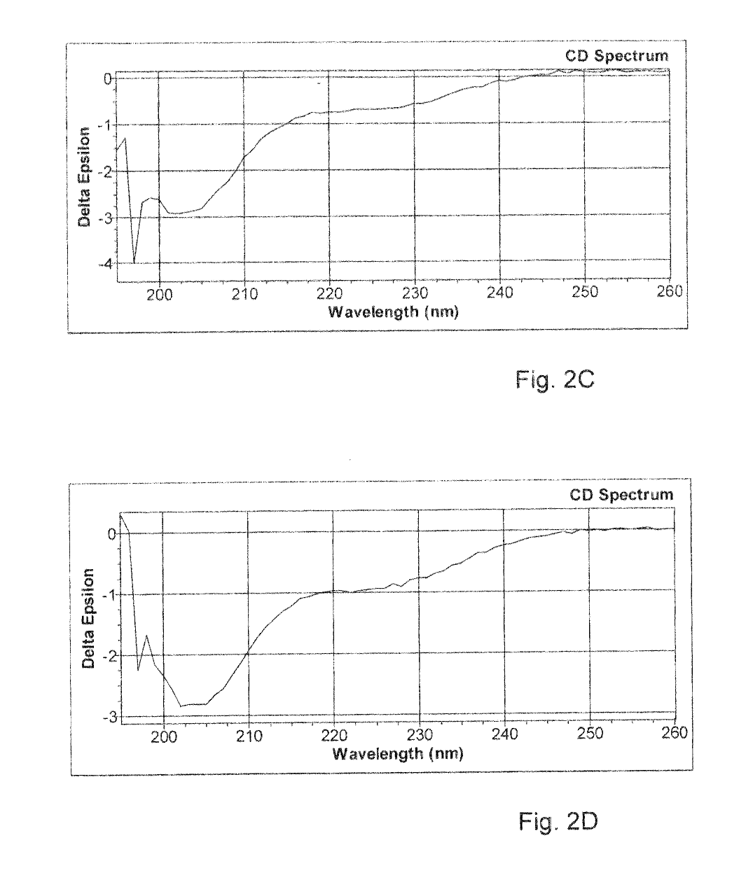

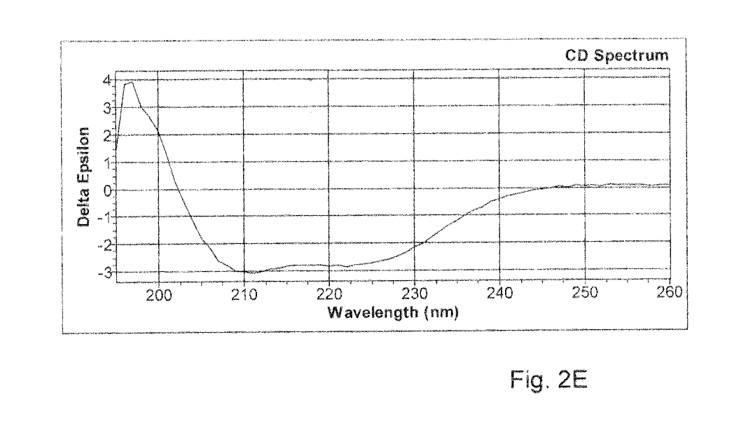

FIG. 2: shows far UV CD spectra of conglutin before and after modifications with A; Native, untreated conglutin, B; RA treated conglutin, C; RAU treated conglutin, D; RAUGA treated conglutin and E; GA treated conglutin. CD spectra were recorded on a J-715 CD spectropolarimeter (Jasco) at 25° C. Samples were measured using a 300 μl quartz cuvette (Hellma) with 0.1 cm path length and a protein concentration of 100 ng/ml was used. CD spectra resulted from averaging twenty repeated scans (step resolution 1 nm, scan speed 100 nm/min) and were buffer-corrected afterwards.

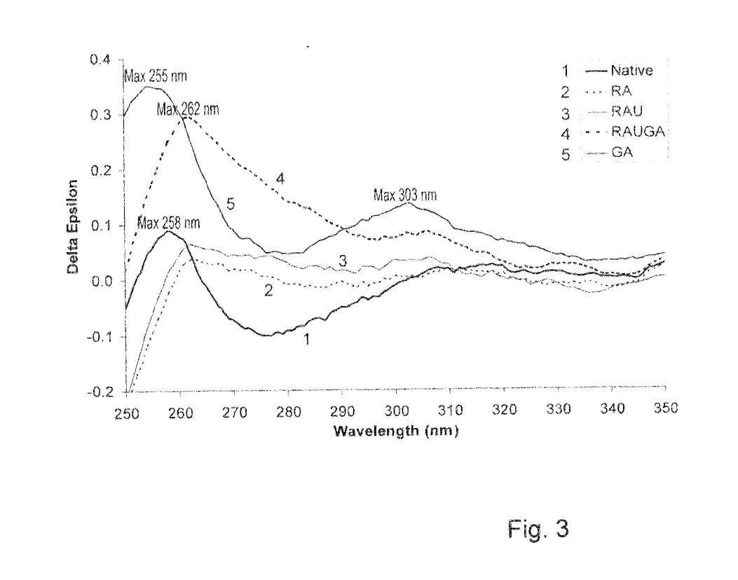

FIG. 3: shows near UV CD spectra of conglutin before and after modifications. Near UV CD spectra of native conglutin (black line), RA treated conglutin (dashed grey line), RAU treated conglutin (dashed black line), RAUGA treated conglutin (dark grey line) and GA treated conglutin (grey line). CD spectra were recorded on a J-715 CD spectropolarimeter (Jasco) at 25° C. Samples were measured using a 300 μl quartz cuvette (Hellma) with 0.1 cm path length, a protein concentration of 500 μg/ml was used. CD spectra resulted from averaging twenty repeated scans (step resolution 1 nm, scan speed 100 nm/min) and were buffer-corrected afterwards and in addition baseline-corrected and smoothed.

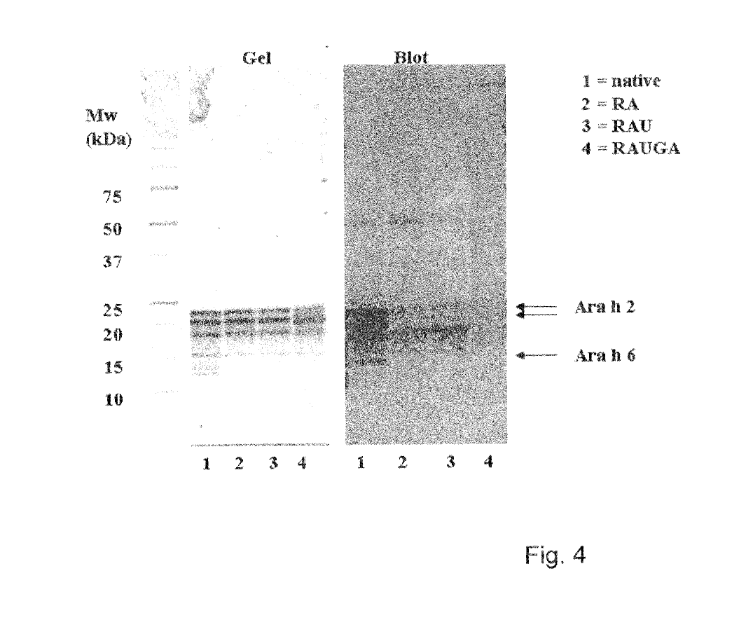

FIG. 4: shows double modification of peanut conglutinin (RA+GA). SDS-PAGE pattern (left panel) and IgE blot (right panel) of the native and treated Ara h2/Ara h6 preparation. Marker proteins (Mw) are indicated on the left, both the gel and the blot contain lanes with native conglutinin (1), reduced and alkylated conglutinin (2), reduced, alkylated conglutin with the addition of urea (3) and reduced, alkylated conglutin with urea and treated with glutaraldehyde (4).

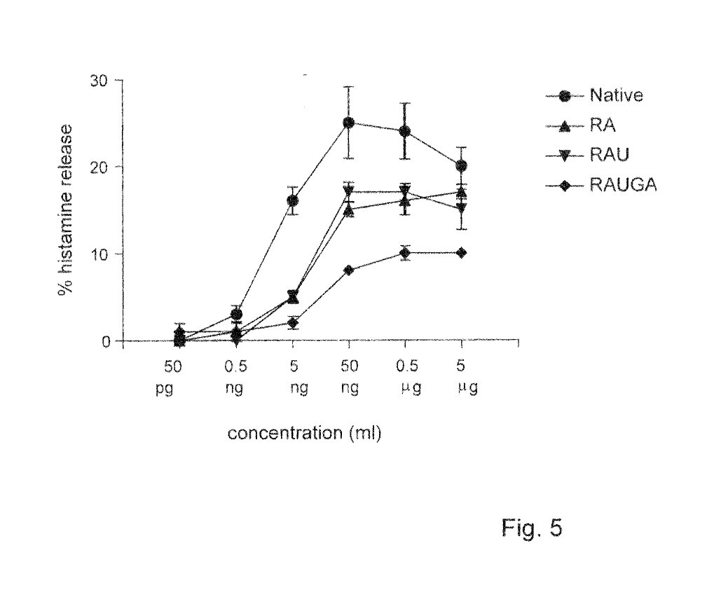

FIG. 5: shows graphic illustration of an example of the percentage histamine release from a peanut-allergic patient. Basophils are stripped from IgE, re-loaded with IgE from the allergic patient and stimulated with peanut allergoid or extract (triplicates). The presented results (% histamine release) are corrected for background/blank.

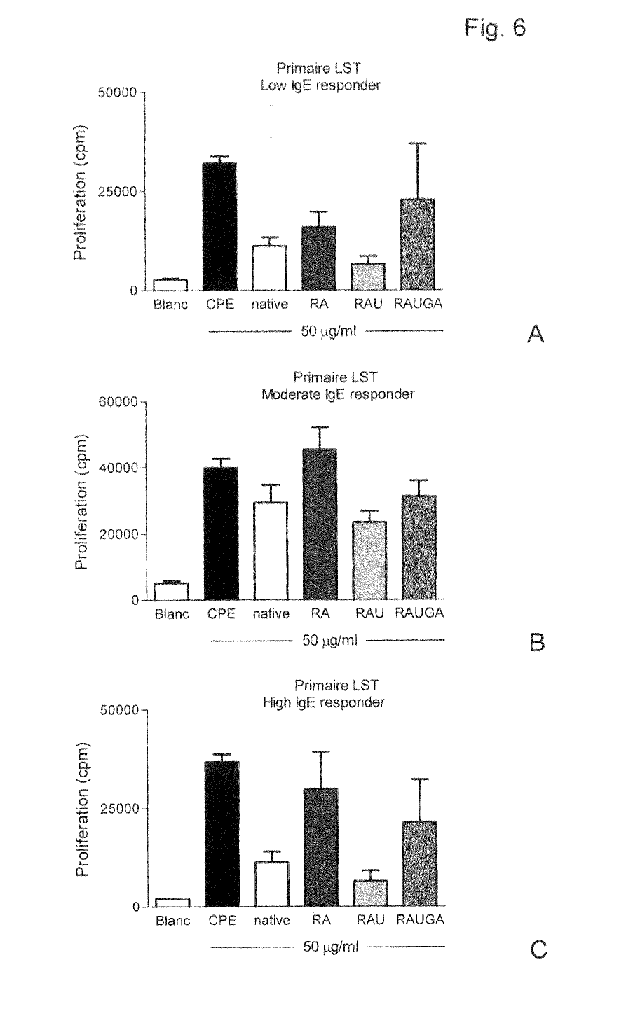

FIG. 6: shows primary LST responses to crude peanut extract (CPE), native (combination Ara h2 and Ara h6) and modified Ara h2/Ara h6 (RA, RAU, RAUGA). Triplicate cultures of a mild (A), moderate (B) and highly peanut-allergic patient (C) were stimulated with 50 μg/ml of allergen or allergoid. Cells cultured in medium were served as control. Six days later, the cultures received an 18-hour pulse of 1 uci per well of thymidine. Cells were harvested, and the incorporated radioactivity was counted the results are expressed as counts per minute.

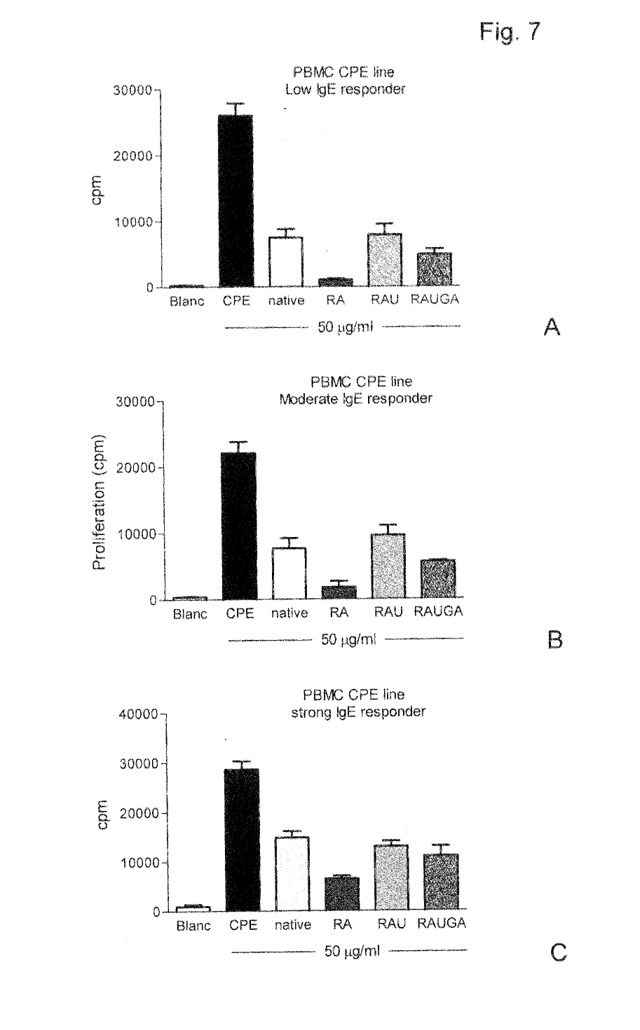

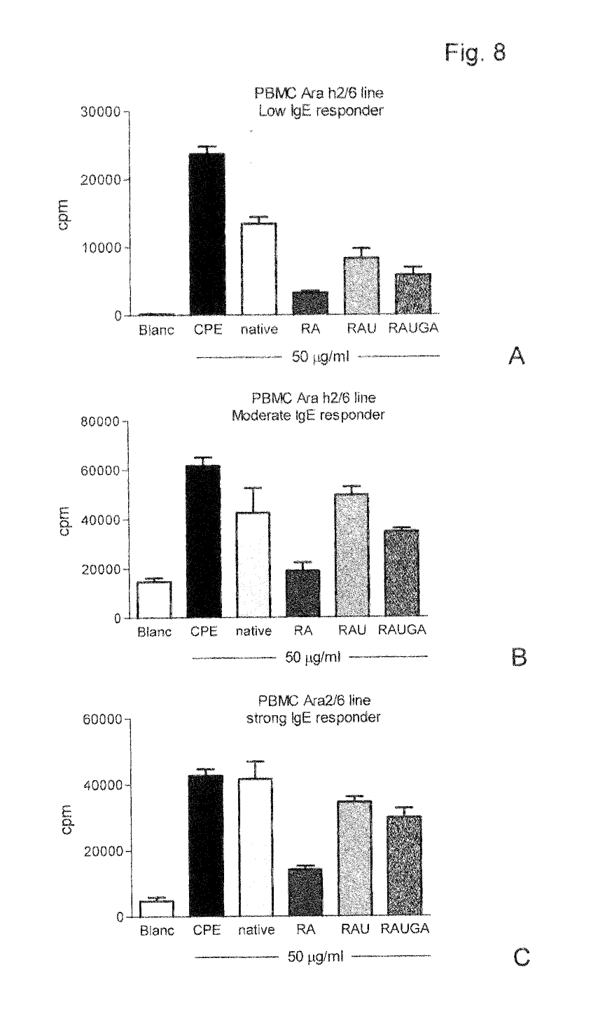

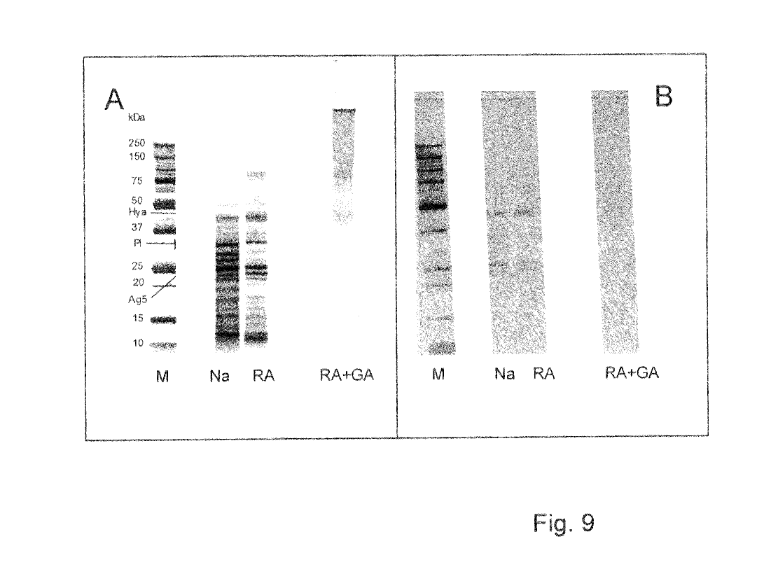

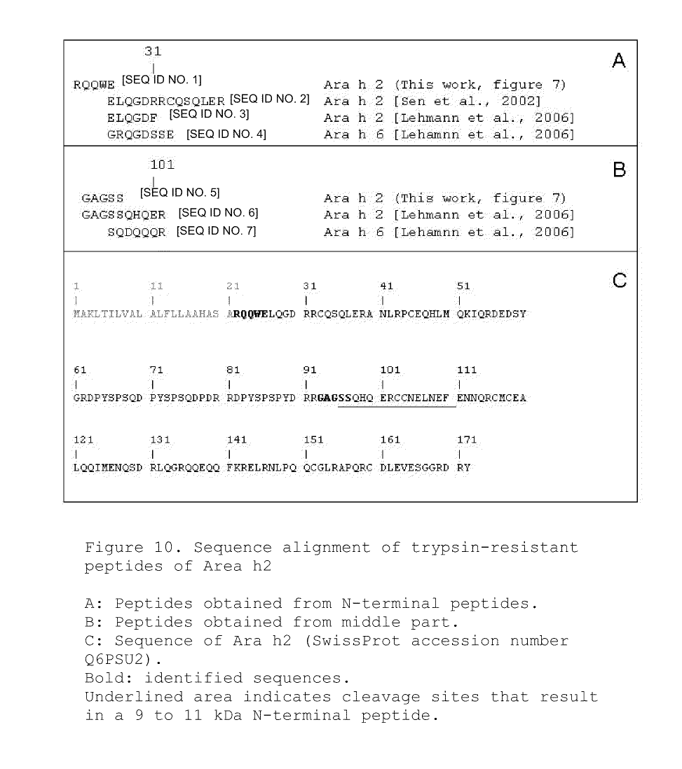

FIG. 7: shows fresh crude peanut extract (CPE)-specific PBMC responses to CPE, native (combination Ara h2 and Ara h6) and modified Ara h2/Ara h6 (RA, RAU, RAUGA). Triplicate cultures of a mild (A), moderate (B) and highly peanut-allergic patient (C) were stimulated with 50 μg/ml of allergen or allergoid. Cells cultured in medium were served as control. Six days later, the cultures received an 18-hour pulse of 1 uci per well of thymidine. Cells were harvested, and the incorporated radioactivity was counted the results are expressed as counts per minute.

FIG. 8: shows fresh Ara h2/Ara h6-specific PBMC responses to crude peanut extract (CPE), native (combination Ara h2 and Ara h6) and modified Ara h2/Ara h6 (RA, RAU, RAUGA). Triplicate cultures of a mild (A), moderate (B) and highly peanut-allergic patient (C) were stimulated with 50 μg/ml of allergen or allergoid. Cells cultured in medium were served as control. Six days later, the cultures received an 18-hour pulse of 1 uci per well of thymidine. Cells were harvested, and the incorporated radioactivity was counted the results are expressed as counts per minute.

FIG. 9: shows double modification of wasp venom (RA+GA). SDS-PAGE pattern (panel A) and IgE blot (panel B) of the native and treated wasp venom. Marker proteins (M) are indicated on the left, both the gel and the blot contain lanes with native wasp venom (Na), reduced and alkylated wasp venom (RA) and reduced, alkylated and glutaraldehyde treated wasp venom (RA+GA).

FIG. 10: Sequence alignment of trypsin-resistant peptides of Area h2
A: Peptides obtained from N-terminal peptides.
B: Peptides obtained from middle part.
C: Sequence of Ara h2 (SwissProt accession number Q6PSU2).
Bold: identified sequences.
Underlined area indicates cleavage sites that result in a 9 to 11 kDa N-terminal peptide [SEQ ID NOs: 1-8].

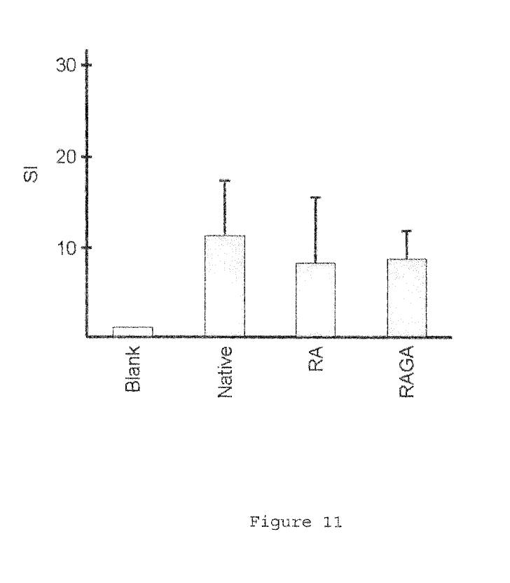

FIG. 11: shows T-cell reactivities of native and modified (RA and RAGA) Ara h2 and Ara h6 preparations derived from a natural source.

### EXAMPLES

### Example 1

### Peanut Protein Extraction and Purification

Peanut extract was prepared using commercially available peanut meal. Purified Ara h1 and Ara h 2 are available at TNO (Zeist, The Netherlands) and described in detail by Koppelman et al., Clin. Exp. Allergy, April 2005, 35(4):490-7.

In short, lyophilized crude peanut extract (CPE) was dissolved in 20 mM TRIS-bis-propane, pH 7.2 (TBP) to a final concentration of 1 mg/mL. Undissolved particles were removed by centrifugation (3000×g, 15 min) and the solution was applied on a 8 mL Source Q column (1×10 cm) previously equilibrated with TBP. After washing the column with 80 mL of TBP, a linear gradient of 200 mL (0-1 M NaCl in TBP) was applied to elute the bound proteins (2 mL/min). To remove traces of peanut lectin from Ara h2 (less than 1%), Ara h2 was dialysed against 50 mM NaAc, pH 5.0 and loaded on a 1 mL Source S column (0.5×5 cm) equilibrated with 50 mM NaAc, pH 5.0. After washing with 10 mL of 50 mM NaAc, pH 5.0, the column was eluted using a 25 mL linear gradient (0-500 mM NaCl in 50 mM NaAc, pH 5.0) with a flow velocity of 0.25 mL/min.

Ara h6 was purified according to earlier described procedures (Koppelman et al., Clin. Exp. Allergy, April 2005, 35(4):490-7), ammonium sulphate was added to the crude extract to attain a concentration of 40% saturation at 4° C. The solution was centrifuged (45 min, 8000×g, at 4° C.). The cold supernatant was filtered over glass wool to remove fat particles. Ammonium sulphate was then added to a concentration of 80% saturation at 4° C. The solution was centrifuged again (45 min, 10,000×g, at 4° C.). The pellet was then resuspended in 1.3 L 20 mm Tris/HCl pH 8.0 containing 1 mm EDTA. This preparation is referred to as concentrate. The nearly clear concentrate was filtered using a G3 glass filter and the filter was washed with 80 mL of 20 mm Tris/HCl pH 8.0 containing 1 mm EDTA and 1380 mL clear filtrate was obtained. A fraction of 230 mL was applied on a Sephadex (Pharmancia, Uppsala, Sweden) G75 column (7200 mL column volume, diameter 20 cm, height 23 cm) and eluted with 20 mm Tris, pH 8.0 at 100 mL/min.

Fractions containing the target protein (3200-5700 mL) were immediately further processed using anion exchange chromatography. All steps until anion exchange chromatography were performed at 4° C. The enriched fractions of the 12 Sephadex G75 runs were combined, warmed up to 25° C. and applied to a 3600 mL Source 15Q (Pharmancia) column (diameter 20 cm, height 12 cm) previously equilibrated with 20 mm Tris, pH 8.0 (loading buffer).

After washing with loading buffer, the column was eluted with a 40 L salt gradient of 0-0.25 mM NaCl in loading buffer at a flow of 100 mL/min. Fractions of 400 mL were collected and analysed for Ara h 6 content and purity. Purified Ara h 6 was stored in small portions at −20° C. All buffers used were filtered through 0.45 μm Durapore membranes (Millipore, Bedford, Mass., USA).

Conglutin is the protein fraction of a peanut kernel comprised of mainly 3 isoforms called Ara h 2 (2 isoforms) and Ara h 6 (1 isoform). Peanut conglutin can be prepared by extracting ground peanut meal, precipitation with ammonium sulphate, and subsequent size exclusion chromatography as described by Koppelman et al., Clin. Exp. Allergy, April 2005, 35(4):490-7. Protein concentrations in extracts were measured with Bradford analysis (BioRad Laboratories, Hercules, Calif., USA) using bovine serum albumin as a standard.

### Peanut Modifications

### 1. Glutaraldehyde Modification of Ara h1 and Ara h2

Modification with glutaraldehyde was performed by adding a glutaraldehyde to a peanut extract or purified Ara h1 or Ara h2 at different pH values (Tables 2-4). After a 4 hours incubation at room temperature, the modified extract was diafiltrated against buffer over a 5 kD membrane. After diafiltration glycine was added to react with residual aldehyde groups. After a second diafiltration against buffer the samples were stored at 2-8° C. until analysis.

2. Modification of Peanut Conglutin with DTT, Iodoacetamide and Glutaraldehyde

Conglutin was diafiltered and diluted in 5 ml of 100 mM TRIS in absence or presence of 8 M Urea (pH=8.5) at a concentration of 0.5 mg/ml. 0.05 ml of 1M DTT was added and the mixture was incubated for one hour at 56° C. Then, 0.6 ml of 0.5 M Iodoacetamide was added and incubated for 1.5 hours at room temperature. The mixture was diafiltered into 50 mM phosphate buffer (pH=8.0) and the conglutin concentration was readjusted to 0.25 mg/ml in 10 ml. 0.02 ml of a 5% solution of glutaraldehyde was added, and then the mixture was gently shaken overnight at room temperature. An excess of glycin was added to stop the reaction, and the mixture was diafiltered to remove excess of reagents. Instead of 8 M Urea 6M guanidine may be used.

Summarizing, the following samples were prepared:
Untreated conglutin, called native
Reduced and alkylated conglutin, called RA
Reduced and alkylated conglutin prepared in the presence of urea, called RAU
Reduced and alkylated conglutin prepared in the presence of urea, treated afterwards with glutaraldehyde, called RAUGA

### Wasp Venom Modifications

Wasp venom was diafiltered and diluted in 5 ml of 100 mM TRIS containing 8 M Urea (pH=8.5) at a concentration of 0.5 mg/ml. 0.05 ml of 1M DTT was added and the mixture was incubated for one hour at 56° C. Then, 0.6 ml of 0.5 M Iodoacetamide was added and incubated for 1.5 hours at room temperature. The mixture was diafiltered into 50 mM phosphate buffer (pH=8.0) and the wasp venom concentration was readjusted to 0.25 mg/ml in 10 ml. 0.02 ml of a 5% solution of glutaraldehyde was added, and then the mixture was gently shaken overnight at room temperature. An excess of glycine was added to stop the reaction, and the mixture was diafiltered to remove excess of reagents. Instead of 8 M Urea 6M guanidine may be used.

### Circular Dichroism Spectroscopy

CD spectra were recorded on a J-715 CD spectropolarimeter (Jasco) at 25° C. Samples were measured using a 300 μl quartz cuvette (Hellma) with 0.1 cm path length. For far-UV CD measurements (260-195 nm), a protein concentration of 100 mg/ml was used. In case of near-UV CD measurements (350-250 nm), a protein concentration of 500 mg/ml was used. All CD spectra resulted from averaging twenty repeated scans (step resolution 1 nm, scan speed 100 nm/min) Whereas far-UV spectra were only buffer-corrected, near-UV spectra were buffer-corrected and in addition baseline-corrected and smoothed. Far-UV CD spectra were analysed using the program CDNN (CD Spectra Deconvolution, Version 2.1, Böhm, 1997) to predict the secondary structure content of the protein samples.

### Patients

Blood was collected from six peanut-allergic patients, who were included in the study who were previously well-characterized at the department of Dermatology/Allergology (Utrecht, NL). The study was approved by the Ethics Committee of the University Medical Center Utrecht. All patients gave written informed consent. Inclusion criteria were: >18 years of age, peanut allergy proven by double-blind placebo-controlled food challenge (DBPCFC) or by a clear history, and previously determined peanut-specific IgE>3.5 kU/L (preferably>17 kU/L). Previous immunoblot data showed IgE recognition of both Ara h2 and Ara h6 in all patients. Peanut-specific IgE was determined again by CAP upon inclusion in the study. Clinical characteristics of the patients are summarized in Table 1.

TABLE 1
Patient characteristics

| | | | | | | | Previous | Previous |
|---|---|---|---|---|---|---|---|---|
| | | Inclusion | Previous | | Previous | | immuno- | immuno- |
| | | IgE | IgE | Previous | threshold | | blot | blot |
| Patient nr. | Code | peanut | peanut | Müller* | DBPCFC | | Ara h2 | Ara h6 |
| HAL1 | 267979 | >100 | >100 | 4 | 100 | mg | >+++ | >+++ |
| HAL2 | 6068671 | 25.6 | 44 | 4 | 0.1 | mg | +++ | +++ |
| HAL3 | 2915683 | 47.7 | 85 | 4 | 0.1 | mg | >+++ | >+++ |
| HAL4 | 134967 | 78.8 | >100 | 3 | 0.1 | mg | >+++ | >+++ |
| HAL5 | 4378052 | 9.25 | 18 | 4 | 10 | mg | ++ | ++ |
| HAL6 | 2305667 | 14.8 | 18 | 0 | 10 | mg | ++ | ++ |

*Müller score (most severe symptoms by history): 0, symptoms of the oral cavity; 3, respiratory symptoms; 4, cardiovascular symptoms.

### Immunoblot

IgE recognition of the allergen variants was analyzed by IgE immunoblotting. SDS-PAGE gel electrophoresis and IgE immunoblotting was performed using 15% acrylamide gels. Pre-stained molecular weight markers with molecular weights of 14.3, 21.5, 30, 46, 66, 97.4 and 220 kDa were used as reference. Samples were mixed in a 1:1 ratio with 63 mm Tris buffer (pH 6.8) containing 1% dithiotreitol (DTT), 2% SDS, 0.01% bromophenol blue and 20% (v/v) glycerol and were subsequently boiled for 5 min.

Gels were loaded with 2 μg CPE, and 1 μg of the purified major peanut allergens Ara h2 and Ara h6, as well as the 4 allergen variants. Gels were stained with Coomassie brilliant blue R-250 dissolved in destaining solution (10% HAc (v/v), 5% methanol (v/v) in water). After destaining, gels were scanned with an ImageMaster DTS (Pharmacia, Uppsala, Sweden).

To study the immuno-reactivity of the proteins, SDS-PAGE gels were prepared and the separated proteins were transferred to polyvinyldifluoride sheets (Immobilon-P, Millipore Corp., Bedford, Mass., USA). Membranes were blocked with 3% BSA in wash buffer (50 mm Tris, pH 7.5, containing 0.1% BSA and 0.1% Tween 20) for 1 h at room temperature. Patient serum was diluted 50 times, and IgE bound to the membrane was detected with a peroxidase-conjugated goat-anti-human IgE (Kirkegaard and Perry Limited, Gaithersburg, Md., USA).

Solid-Phase IgE-Binding Test

IgE-binding properties were measured by solid-phase immuno assay (Inhibition ELISA), a method often used for determining the potencies of allergen extracts, for example peanut (Koppelman et al., Biol. Chem. 1999; 274(8):4770-7). Here, a pool of plasma obtained from patients with clinical peanut allergy is used. Dilutions of allergen were pre-incubated with patient plasma in phosphate-buffered saline (PBS) containing 0.1% BSA and 0.05% Tween in a final protein concentrations of 250 μg/ml-2.3 ng/ml and a plasma dilution of 450 fold.

The allergens were allowed to bind to IgE for 1 h at room temperature. Subsequently, this mixture was loaded on an allergen-coated plate. In this way, the remaining free IgE in the mixture is able to bind to the allergens attached to the plate. IgE bound to the allergen-coated wells was then detected using an anti-human IgE antibody conjugated to horseradish peroxidase. The inhibition of IgE binding as a function of the amount of allergen present in the pre-incubation sample reflects the potency of that allergen variant for IgE. Potencies were compared using the parallel line approach.

### Basophil Degranulation

Donor basophils from non-allergic persons: Buffy coats (n=4) were collected at the Blood Bank, National University Hospital of Copenhagen. The Blood Bank has a general ethical approval to hand out buffy coats making sure that the blood donors are anonymous. The buffy coats were initially screened for sensitization against food allergens (n=10) and inhalation allergens (n=10) before included into the study. Only non-allergic, anti-IgE responding buffy coats were used. Donor basophils were semi-purified on lymphopreb (PBMC suspension). Their IgE was removed by a rebounce in pH (down to 3.75 and then back to 7.4) and loaded with IgE from sera from patients described in Table 1 (1 hour sensibilization). The basophils were then stimulated with peanut allergoid or extract (5 dilutions, triplicates) and the released histamine was measured. The presented results (% histamine release) were corrected for background/blank.

### Primary Lymphocyte Proliferation

Leukocyte stimulation tests (LST) are a model for the first contact of the immune system with (foreign) antigens. An LST contains different (white blood) cell. Upon contact with antigens, APC's will present the antigen to T-cells, which subsequently will proliferate. This assay was performed to check whether the PBMCs that are cultured to generate peanut-specific TCL have a good primary peanut-specific response.

Furthermore, the assay provides an impression of the potency of the other allergen extracts to induce primary lymphocyte proliferation. PBMCs were purified from 70 ml venous blood from six peanut allergic patients by Ficoll gradient centrifugation. Cells were cultured (37° C. and 5% CO2) in 96-well round-bottom plates in triplicate (2.10⁵ cells/well) in culture medium (IMDM medium containing 5% human serum (HS), penicillin (100 IU/ml), streptomycin (100 mg/ml), and glutamine (1 mmol/ml)) in the presence and absence of CPE, purified Ara h2 and Ara h6, or the 4 allergen variants (all at 50 μg/ml). After 6 days of culture, supernatants were taken for measurements of cytokines (IL-10, IL-13, IFN-γ, TNF-α).

For proliferation, [3H]-TdR (0.75 μCi/well) was added at day 6 for overnight incubation, cells were harvested and incorporation of [3H]-TdR was measured using a 1205 β-plate counter (Wallac, Turku, Finland) and expressed as counts per minute (cpm). Proliferation is expressed as stimulation index (SI; proliferation to allergen stimulation divided by blank). It is desired that the ratio of the SI of the modified protein to the SI of the unmodified protein is as high as close as possible to 1. An SI>2 is considered positive. PBMCs that were left were stored in liquid nitrogen.

### Peanut-Specific T Cell Lines (TCLs)

To be even more specific, (short) T-cell lines can be prepared by culturing with isolated allergens such as Ara h 2 and Ara h 6. Proliferation is considered to be a measure for immunogenicity required for effective immunotherapy. PBMCs were cultured in 48-well flat-bottom plates in triplicate (10⁶ cells/well) in culture medium in the presence of CPE (50 μg/ml), or a mixture of purified Ara h2 and Ara h6 (both 50 μg/ml). IL-2 was added to the cultures (10 U/ml) at day 7.

At day 11, TCLs were restimulated in two wells in a 24-well flat-bottom plate with feedermix containing irradiated allogenic PBMCs (2 donors, 5.10⁵ cells/well) and EBV-transformed B-cells (1×10⁵ cells/well), IL-2 (10 IU/ml), and PHA as mitogen (0.5 μg/ml). At day 21, TCLs were tested for antigen-specificity in 96-well round bottom plates (3·10⁴ cells/well) by stimulation with autologous PBMCs (1·10⁵ cells/well) in the absence or presence of CPE, Ara h2, Ara h6, and the 4 allergen variants (all at 100, 50 and 25 μg/ml). After 48 hours, supernatants were taken for cytokine measurements and 0.75 μCi/well of [3H]-TdR was added for overnight incubation.

Cells were harvested and incorporation of [3H]-TdR was measured using a 1205 β-plate counter (Wallac, Turku, Finland) and expressed as counts per minute (cpm). The stimulation index was the measured counts per minute in the presence of antigen divided by the measured counts per minute in the absence of antigen.

### Results

### GA Modification of Peanut Allergens

Modification of peanut allergens with glutaraldehyde reduces the IgE-binding to some extent, but not sufficiently, even though optimization (pH, concentration of reagents, incubation temperature and time, Tables 1, 2, and 3) has been applied to the reaction conditions.

To further investigate the effect of glutaraldehyde treatment on IgE-binding, SDS-PAGE was performed in combination with IgE-blotting using 12.5% gel precast gels (Amersham Biosciences) and PVDF membranes.

FIG. 1 shows the results of purified Ara h1 and purified Ara h2. Modification of Ara h1 results in loss of almost all the individual bands (FIG. 1, lane 3) but not the loss of IgE-binding activity, as the intensity of the bands in lane three is not less than that in lane 2. For Ara h2, no change in molecular weight is observed. Next to the observation that the molecular weight of Ara h2 is not increased significantly upon treatment with glutaraldehyde, the IgE binding of the glutaraldehyde-treated allergens on blot is not decreased. This indicates that modification by glutaraldehyde on the molecular weight of Ara h 2 and IgE-binding is limited.

TABLE 2
Modification results of a whole peanut extract at different
pH's and different concentrations of glutaraldehyde

| | Glutaraldehyde 50% | |
|---|---|---|
| pH | μl per mg protein | Residual activity % |
| 7.2 | 0.6 | 28 |
| 7.2 | 1.2 | 22 |
| 8.0 | 0.6 | 22 |
| 8.0 | 1.2 | 27 |
| 8.0 | 1.8 | 28 |
| 9.0 | 0.6 | 33 |
| 9.0 | 1.2 | 28 |
| 9.0 | 1.8 | 25 |

TABLE 3
Ara h1 modified with glutaraldehyde

| | Glutaraldehyde 50% | |
|---|---|---|
| pH | μl per mg protein | Residual activity % |
| 7.2 | 1.2 | 52 |
| 7.2 | 1.8 | 37 |

TABLE 4
Ara h2 modified with glutaraldehyde

| | Glutaraldehyde 50% | |
|---|---|---|
| pH | μl per mg protein | Residual activity % |
| 7.2 | 1.2 | 40 |
| 7.2 | 1.8 | 44 |

### Combined RA and GA Modification of Peanut Allergens

As expected, RA treatment resulted in a loss of Cys residues as determined with standard amino acid analysis (Table 5). GA treatment after RA treatment resulted in a loss of Lys residues as determined with standard amino acid analysis, which is unexpected because GA treatment alone (FIG. 1, Tables 1-3) did not result in sufficient modification of the Ara h2 on a functional level (IgE-binding):

TABLE 5
Degree of modification after RA and GA treatment

Modified amino acids (%)

| | Cys | Lys |
|---|---|---|
| 1. Untreated | 0 | 0 |
| 2. RA | 99 | 0 |
| 3. RAU | 69 | 0 |
| 4. RAGA | 85 | 55 |
| 5. RAUGA | 88 | 59 |

Characterization of Peanut Protein Structure after RA and GA Modification

### Secondary Protein Folding Level

The consequences of modification and double modification were investigated on the level of secondary protein structure. Far UV circular dichroism spectra were recorded (195-260 nm) and the percentages of secondary structure elements were calculated.

FIG. 2 shows the individual far UV CD spectra and Table 6 summarizes the corresponding secondary structure elements. From FIG. 2 it is clear that RA modification in presence or absence of urea, and with or without GA treatment results in a dramatic change of the spectrum.

The fact that the ellipticity around 220 nm has decreased is indicative for loss of helical structures and formation of random coil (denaturation of protein). Furthermore, the minimum has shifted to approx. 205 nm which is indicative for the formation of beta-structure. These findings were supported by the calculations on the secondary structure (Table 6).

TABLE 6
Secondary structure elements of conglutins
after RA and/or GA treatment

| | Reference | RA | RAU | RAUGA | GA |
|---|---|---|---|---|---|
| Helix | 34.30% | 17.30% | 16.70% | 17.90% | 31.60% |
| Antiparallel | 8.10% | 16.90% | 17.40% | 16.40% | 8.90% |
| Parallel | 8.70% | 14.20% | 14.70% | 14.00% | 9.40% |
| Beta-turn | 16.60% | 21.20% | 21.40% | 21.00% | 17.10% |
| Random coil | 32.70% | 42.40% | 43.40% | 42.30% | 34.60% |
| Total sum | 100.30% | 112.10% | 113.60% | 111.50% | 101.50% |

While the modification of peanut conglutin with only GA does not result in a change of secondary structure, RA treatment reduces the helical content resulting in an increase of random coil and beta-structure. These protein transformations have been observed earlier for Ber e 1, a conglutin-like protein from Brazil nut [Koppelman et al., J. Agric. Food Chem., 2005, 53(1), pp. 123-31].

### Tertiary Protein Folding Level

The consequences of modification and double modification were also investigated on the level of tertiary protein structure by near UV circular dichroism (250-350 nm). FIG. 3 shows the near UV CD spectra of native and modified conglutin. The fact that RA and RAU modified conglutin spectra (dashed lines) show hardly any ellipticity confirms denaturation of the protein (formation of random coil).

Native, GA treated and RAUGA treated protein all show ellipticity with absorption maxima at 258, 255 and 262 nm, respectively. These maxima are indicative for phenylalanine (250-270 nm) and alterations in its environment.

Conglutin contains three phenylalanins and one of them is located in a helix next to a lysine. The binding of GA to this lysine in case conglutin is treated with GA appears to change the environment of phenylalanine resulting in a shift of the absorption maximum (from 258 to 255 nm). As described above, spectra of RA and RAU modified conglutin did not show any signal.

The use of GA after reduction-alkylation seems to regain asymmetry in the area of phenylalanine as an absorption maximum at 262 nm was observed in the RAUGA spectrum. This maximum differs from the maxima observed in the spectra of native and GA-treated conglutin which means that the environment of the phenylalanine in these three samples differs. No signals of tyrosine (270-290 nm) and tryptophan (280-300 nm) in all spectra can be explained by the fact that these residues are located in random coils of the protein.

### IgE Binding Properties of Modified Peanut Conglutin

### Solid-Phase IgE-Binding Test

IgE-binding properties were measured by solid-phase immuno assay using a pool of serum obtained from patients with clinical peanut allergy. A sample with unchanged IgE-binding properties would have a potency of 100%. A sample in which no IgE-binding is left would measure 0%. Table 7 shows the potencies for differently treated samples. The relative potency of the modified product is in all 3 cases lower than 1%. However, the RAUGA variant shows an even lower IgE-binding property compared to RA and RAU.

TABLE 7
IgE-binding potencies of conglutins after RA and GA treatment

| Sample | Potency |
|---|---|
| Native | 100% |
| RA | 0.6% |
| RAU | 0.6% |
| RAUGA | 0.1% |

### SDS-PAGE and IgE-Immunoblotting

The molecular weight of conglutin is not affected substantially upon RA treatment. The presence of urea or modification with GA does not change the molecular weight (FIG. 4). To further substantiate the IgE-binding properties, IgE-immunoblotting combined with SDS-PAGE was performed. RA and RAU already have low IgE-binding (FIG. 4, right panel, lane 2 and 3), and RAUGA reduced IgE-binding even stronger (FIG. 4, right panel, lane 4) and no immune response could be detected on the blot.

IgE-blots were repeated with individual patient sera, and IgE binding was scored semi-quantitatively (Table 8). Residual IgE binding to RA and RAU were 30 to 70%, while for RAUGA 0-10%, illustrating the added value of the double modification.

TABLE 8
IgE-binding potencies of conglutins after RA and GA treatment,
individual patient sera used in (semi-quantitative) IgE-blot

| | Ara h2 | | | | Ara h6 | | | |
|---|---|---|---|---|---|---|---|---|
| Patient | native | RA | RAU | RAUGA | native | RA | RAU | RAUGA |
| HAL1 | 100% | 70% | 60% | 10% | 100% | 70% | 60% | 10% |
| | (5+) | | | | (5+) | | | |
| HAL2 | 100% | 60% | 30% | 0% | 100% | 10% | 5% | 0% |
| | (3+) | | | | (3+) | | | |
| HAL3 | 100% | 60% | 60% | 10% | 100% | 10% | 10% | 0% |
| | (3+) | | | | (3+) | | | |
| HAL4 | 100% | 70% | 50% | 10% | 100% | 70% | 40% | 0% |
| | (4+) | | | | (4+) | | | |
| HAL5 | 100% | 60% | 60% | 5% | 100% | 40% | 40% | 0% |
| | (2+) | | | | (2+) | | | |
| HAL6 | 100% | 60% | 40% | 10% | 100% | 30% | 10% | 0% |
| | (3+) | | | | (3+) | | | |
| Mean | 100% | 63% | 50% | 8% | 100% | 38% | 28% | 2% |
| | (3.3+) | | | | (3.3+) | | | |

### Basophil Histamine Release by Modified Peanut Allergens

The potency of the 6 different sera (strength in sensitizing basophils) to the 4 allergen variants was tested. RA, RAU and RAUGA are poorer in inducing a histamine release from donor basophils sensitized with serum from peanut allergic patients. In FIG. 5, an example of histamine release for one of the patients is shown. Native conglutin induces histamine release (HR) already at low concentrations. RA and RAU show a similar decreased ability to induce HR.

Unexpectedly RAUGA induces even less HR, as observed by a later onset of HR and a lower plateau. Concentrations required for 10% HR, which is considered a relevant threshold for histamine release, are for RA-treated conglutin between 5 and 50 ng/ml, and for RA-treated conglutin treated with GA afterwards between 500 and 5000 ng/ml, a hundred fold higher, indicating a hundred fold decrease in potency.

### T Cell Proliferation

Data are given for three types of peanut allergic patients: With low peanut specific IgE, with moderate, and with high peanut specific IgE.

### Leukocyte Stimulation Test (LST)

All donors (6/6) had a strong proliferation upon CPE stimulation. Data for 3 different patients (With low peanut specific IgE, with moderate, and with high peanut specific IgE) are shown in FIG. 6.

The response to Ara h2 was weaker in stimulation index (SI) than the response to Ara h6. It is noted that RA treatment results in a higher proliferation than native, the response to RAUGA was comparable and the response to RAU was lower as compared to native extract. Considering that the same proteins are present, and in the same concentration, another factor must cause the enhanced proliferation. Probably, the increased digestibility of conglutins after reduction and alkylation explains this.

Increased digestibility may turn conglutins into better substrates for antigen-presenting cells. In contrast to what has often been described for GA treatment of allergens, in this case, after RA treatment, treatment with GA does not result in decreased proliferation.

### CPE-Specific T-Cells

The analysis of the T cell response to a specific allergen is more specific and more optimal after pre-selection of allergen-specific T cells by the generation of short-term TCLs. Therefore, short-term TCLs were generated specific for CPE. Data for 3 different patients (With low peanut specific IgE, with moderate, and with high peanut specific IgE) are shown in FIG. 7.

It is noted that RA treatment results in a lower proliferation than native. Surprisingly, treatment with GA after RA restores the proliferative responses of RA treated sample. This effect is most pronounced for the patient with the lowest peanut-specific IgE. In contrast to what has often been described for GA treatment of allergens, in this case, after RA treatment, treatment with GA does not result in decreased proliferation, but in an improved proliferation. It is also interesting to note the RA treatment in the presence of Urea (RAU) results in a higher T-cell proliferation in this model system.

### Ara h 2/Ara h 6-Specific T-Cells

Data for 3 different patients (With low peanut specific IgE, with moderate, and with high peanut specific IgE) are shown in FIG. 8.

The data support the observations of the previous mentioned T-cell data obtained with T-cells for peanut extract. All TCLs had a very low background proliferation, with high proliferation upon CPE stimulation. This shows that, indeed, the generation of peanut-specific TCLs enhanced the peanut-specific response (mean SI to CPE around 50) as compared to the primary response (mean SI to CPE around 12). None of the TCLs responded to parvalbumin, which was included to check the TCLs for peanut-specificity.

### Modification of Wasp Venom by RA and GA Treatment

The IgE-binding potency was determined as for conglutin, using in the present case a pool of sera from wasp venom-allergic patients. Table 9 shows the results.

TABLE 9
Potency of wasp venom sample treated with RA and GA

| Sample | Modification applied | Potency |
|---|---|---|
| Native | None | 100% |
| RA | Reduced and alkylated | 14% |
| RAUGA | Reduced and alkylated, and | 0% |
| | glutaraldehyde treated | |
| GA | glutaraldehyde treated | n.a. due to protein precipitation |

FIG. 9 shows the SDS-PAGE pattern and IgE blot of the native and treated wasp venom. It is clear from Table 9 that gluteraldehyde treatment alone, as is common for other allergens, is not suitable for wasp venom under the chosen conditions because of precipitation of the wasp venom. Reduction and alkylation reduces the IgE binding substantially to 14%. Surprisingly, subsequent treatment with gluteraldehyde further decreases the IgE binding without excessive precipitation of the wasp venom.

### Conclusions

Double modification peanut conglutin by RA and GA treatment has been performed for peanut and wasp venom allergens. While the modification of peanut conglutin with GA only does not result in a change of secondary structure, RA treatment reduces the helical content resulting in an increase of random coil and beta-structures. Furthermore, RA treatment followed by GA modification results in a tertiary structure that differs from that of conglutin treated only with RA. It appears that in case of the double modification not only the Cys residues are modified, but also the Lys residues.

Double modification of peanut conglutin by RA and GA treatment leads to a pronounced reduction of IgE binding, also in functional way (basophil histamine release). The additional effect of GA compared to RA alone is surprising because GA treatment alone did not result in substantial decrease of potency. RA conglutin has decreased IgE-binding as compared to native, demonstrated by IgE-ELISA, IgE blot, and BHR. Treatment with GA after RA pronounces this effect up to a hundred fold. This is unexpected because GA treatment without pre-treatment by RA does not decrease IgE binding substantially (only 2-3 fold, FIG. 1). Our data show that all 3 tested modifications lead to a reduction in IgE binding, with the strongest reduction observed after both reduction/alkylation and glutaraldehyde treatment (RAUGA).

The double modification of wasp venom also results in a strongly diminished IgE-binding, far more pronounced that RA treatment alone. This was surprising because GA treatment without preceding RA treatments was not successful due to precipitation.

T cell proliferation tests were performed where PBMC responses can be affected by the presence of multiple cell types and therefore the clearest conclusions can be drawn from the data obtained with the antigen-specific TCLs. For immunotherapy, the best option would be a modification which leads to (near-) complete reduction of IgE-binding, and maintenance of T cell responses which is needed for immunomodulation. From the 3 modified peanut proteins, RAU induced a good T cell response whereas IgE binding was reduced substantially as described above. The IgE binding to RA was slightly less reduced than to RAU, and the T cell response was less strong, which suggest that this modification is less optimal for application in SIT. IgE-binding to RAUGA was reduced almost completely and RAUGA also induced a strong T cell response. In that respect, RAUGA would be the best candidate. For venom allergens, the effect of the double modification with RA and GA on IgE-binding has been evaluated. While GA treatment alone results in protein precipitation, pretreatment with RA leads to an almost complete reduction of IgE binding, while the proteins remained soluble.

### Example 2

### Materials and Methods

### Test Proteins

Crude peanut extract (CPE) was prepared from ground peanut (Arachis hypogaea, variety: Runner) as described earlier [Koppelman et al., 2001]. Ara h1, Ara h2, Ara h3, and Ara h6 were purified as described earlier [de Jong et al., 1998; Koppelman et al. 2003, Koppelman et al., 2005]. N-terminal sequencing was performed by Edman degradation, using bands excised from SDS-PAGE gels (SeCU, Utrecht, The Netherlands).

### Proteases

Porcine pepsin was purchased from Sigma (St. Louis, Mo., USA, # P-6887). This product was chosen because it has the highest specific activity commercially available (3300 U/mg for this particular batch), and because other researchers investigating the digestibility behavior of potentially allergenic proteins use this product [Thomas et al, 2004]. Trypsin from bovine pancreas (treated with L-1-Tosylamide-2-phenylethyl chloromethyl ketone (TPCK) to reduce the chymotrypsin activity) was obtained from Sigma (T-1426). The proteases were dissolved immediately before the digestion experiments and used within 15 minutes in order prevent possible loss of activity due to auto-digestion.

### Pepsin Digestion Assay Conditions

Tubes containing 1.52 ml of simulated gastric fluid (SGF) were prepared with the pH adjusted to 1.2 (0.063 N HCl, containing 35 mM NaCl and 4000 U pepsin). In a control experiment the potential effect of adding pepsin and test proteins on the final pH was found to be negligible (less than 0.05 pH points). The SGF was pre-warmed to 37° C. for 5 minutes and 80 μl of 5 mg/ml test protein was added at time point t=0. For CPE, due to a lower solubility, SGF was prepared at a higher concentration such that the addition of 400 μl of 1 mg/ml CPE resulted in the same final concentration of HCl, NaCl, pepsin, and test protein. The ratio of pepsin:substrate protein was 10 U pepsin: 1 μg substrate protein.

Starting with a pepsin specific activity of 3300 U/ml and a substrate protein concentration of 250 μg/ml, 760 μg/ml pepsin was applied. Additionally, pepsin was diluted 10- or 100-fold with respect to the above calculation. Samples of 200 μl were collected at time points: 0.5, 2, 5, 10, 20 30 and 60 min. Digestion was stopped at appropriate times by mixing with 70 μl of 200 mM NaHCO₃ (pH=11.0) and 70 μl of 5 times concentrated electrophoresis buffer [Laemmli et al., 1970] containing 40% glycerol, 20% SDS, with or without 5% β-mercaptoethanol, 0.33 M TRIS (pH 6.8) and 0.05% bromophenol blue.

The samples of 0.5, 1, 2 and 5 min were heated for 5 minutes at >75° C. directly after taking the sample of time point 5 min. Samples of other time points were heated immediately after sampling. All samples were stored at −20° C. until SDS-PAGE analysis.

The sample of time point t=0 min was prepared by adding bicarbonate and Laemmli buffer and heating SGF prior to the addition of test protein. After adding the test protein, the samples were heated again to ensure full denaturation. Potential pepsin auto-digestion was tested by adding 80 μl of water to SGF and incubation of 60 minutes as described for test proteins. Protein stability at low pH was tested by preparing SGF without pepsin, and incubating the test protein for 60 minutes.

### Trypsin Digestion of Ara h2

Lyophilized Ara h2 was dissolved at 1 mg/ml in 65 mM TRIS buffer pH 8.3 containing 1 mM EDTA, and mixed with trypsin such that a final concentration of 0.9 mg/ml Ara h2 was reached. The final concentration of trypsin was adjusted to 7.2 μg/ml, 24 μg/ml, and 72 μg/ml. 50 μl samples were taken at 5, 10, 20, 30, 40, 60, and 90 minutes and were immediately stopped by adding 1/5 volume of 5 times concentrated SDS-PAGE sample buffer (containing 40% glycerol, 20% SDS, 0.33 M TRIS (pH 6.8) and 0.05% bromophenol blue) containing 1% DTT.

To isolate the digestion-resistant peptides, digestion with 0.3 μM trypsin was stopped after 20 minutes by rapid removal of trypsin by means of anion exchange chromatography, followed by PMSF treatment (1 mM) in a boiling water bath for 30 minutes. Digestion-resistant peptides were further separated by size exclusion chromatography after reduction and alkylation of Cys residues as described previously for 2S albumin from Brazil nut [Koppelman et al., 2005a].

### SDS-PAGE

SDS-PAGE was performed essentially according to Laemmli [Laemmli et al., 1970] with the MiniProtean system (BioRad, Richmond, Calif., USA) using manually prepared 15% polyacrylamide gels. A volume of 20 μl per sample, including Laemmli loading buffer, was loaded and electrophoresis was stopped just before the bromophenol blue-containing front reached the end of the gel. Gels were stained in 1% Coomassie Brilliant Blue R-250 (Sigma, St. Louis, Mo., USA) in 50% methanol/20% acetic acid overnight. Subsequently, gels were washed with 50% methanol/20% acetic acid for 5 minutes and destained with 50% methanol/20% acetic acid for 30 minutes. After that, gels were further destained with 25% methanol/10% acetic acid for 2 hours.

### Results and Discussion

### Pepsin Digestion of Crude Peanut Extract

In a first experiment, crude peanut extract was digested with pepsin according to the protocol of Thomas et al. (2004). This protocol was designed to investigate the comparative stability to pepsin of novel proteins. This protocol uses a high pepsin concentration, 760 ng pepsin and 250 ng substrate protein per ml. Taking into account the specific activity of the pepsin, the pepsin:substrate ratio is 10 U/μg, the same as Thomas et al (2004) used.

At time point 0, before adding pepsin, a characteristic peanut extract pattern was found. By 0.25 mins substantial proteolysis of the crude peanut extract was observed. Peptides in the molecular weight region of 10 to 25 kDa originate from this proteolysis, and some remained up to an incubation time of 30 minutes.

Under reducing conditions, clear bands at approximately 10 kDa were visible that correspond to a digestion-resistant fragment of Ara h2. Because Ara h2 is a minor constituent of the peanut, more conclusive results cannot be obtained on individual peanut allergens using a mixture (whole extract) of peanut proteins.

In contrast to the relative stability of the protein bands at approximately 20 kDa, the protein bands at higher molecular weights (63.5 kDa, Ara h1 and 45 kDa, Ara h3) disappeared rapidly. Note that the remaining band at approximately 40 kDa is pepsin and not a peanut protein. Because limitations exist in the interpretation of studies with crude peanut extracts, digestion experiments were repeated with the purified individual allergens Ara h1, Ara h2, Ara h3 and Ara h6.

### Pepsin Digestion of Individual, Major Peanut Allergens

### Ara h1

SDS-PAGE analysis revealed that the Ara h1 band from CPE disappeared quickly upon digestion with pepsin. Therefore digestion of purified Ara h1 was also conducted with lower concentrations of pepsin. As expected, lowering the pepsin concentration resulted in a more gradual breakdown of peptides. When pepsin was applied in a 100-fold lower concentration as compared to the protocol described by Thomas et al. (2004), resulting in 0.1 U of pepsin per μg substrate, peptides between approximately 20 and 50 kDa appeared.

The analysis on SDS-PAGE did not show differences between reduced and non-reduced samples, as expected based on the fact that Cys residues are not involved in intra- or intermolecular disulfide bridges for Ara h1. The trimeric and the oligomeric organization of Ara h1 on the quaternary folding level is not supported by disulfide bridges and the denaturing conditions of SDS result in dissociation of these multimers. This explains why such multimers are not seen present on SDS-PAGE.

Prior art investigated the pepsin-induced hydrolysis of Ara h1 and applied a 20-lower concentration as compared to the protocol of Thomas et al. (2004). Some peptides of approximately 5 and 10 kDa, stable for 2-8 minutes, were found in line with the data published showing a protein band at 10 kDa stable for up to 2 minutes, and a protein band at 6 kDa stable for up to 8 minutes.

Another publication described the digestion of Ara h1 with low pepsin concentration, comparable to the present lowest concentration. Analysis was performed using size exclusion chromatography under denaturing conditions in order to exclude association of peptides by interactions that support the protein structure on the tertiary and quaternary folding levels. Peptides of relatively high molecular weight, e.g. approximately half of the mass of the intact Ara h 1 monomer were found as judged on their chromatograms.

This is consistent with the present observations of Ara h1 treated with the lowest concentration of pepsin. The digestibility of Ara h1 was described using pepsin and applied immunoblotting with a monoclonal antibody to detect proteolytic breakdown products. The absence of any bands on their blot could either be explained by the fact that Ara h1 was degraded rapidly, or by the loss of immuno-reactivity of Ara h1 after only limited digestion. The present experiments on the digestion of Ara h1 confirm earlier work that it is rapidly digested by the high concentrations of pepsin.

### Ara h3

A first hint of the relatively high digestibility of Ara h 3 is found on SDS-PAGE, where the bands of the acidic and basic subunits of Ara h3 disappear quite rapidly from CPE. The band of pepsin which migrates at a similar molecular weight as the acidic subunits of Ara h 3 makes it difficult to interpret the fate of this subunit.

The digestion of purified Ara h3 showed migration of the pepsin band at the same molecular weight level as Ara h3, but under non-reducing conditions, the acidic and basic subunit were still associated by a disulfide bridge, giving rise to several bands at approximately 70 kDa, [Piersma et al., 2005].

Reducing conditions showed dissociation of the subunits giving rise to the typical pattern for Ara h3 with heterogeneity in the N-terminal acidic subunit [Koppelman et al., 2003; Piersma et al., 2005].

Pepsin digestion was rapid using the conditions applied by Thomas et al. (2004) where all Ara h3 was hydrolyzed after 0.25 minutes. The remaining peptide at approximately 10 kDa disappeared after 1-2 minutes. However, at the lowest concentration of pepsin (100-fold lower; 0.1 U of pepsin per μg Ara h 3) some peptides of intermediate weight (10-30 kDa) remained but for only 2-4 minutes. Little work has been reported by others on the digestion of Ara h3. Ara h3 digestion with pepsin (pepsin:protein ratio=1:500 (w/w) was described earlier. In this study, they used the same pepsin as is used in the current study (Sigma P-6887), with a similar specific activity. The 1:500 w/w therefore corresponds to 0.07 U/μg Ara h3, about a two-fold lower ratio than the present lowest concentration.

They analyzed by size exclusion chromatography, under reducing and denaturing conditions, allowing a comparison of molecular weight with SDS-PAGE (reducing conditions) analysis. After 10 minutes, the majority of the protein was found in the range of 7 to 14 kDa, and after 60 minutes the majority of the peptides were <7 kDa. This remaining fraction may be explained by the lower pepsin concentration in the digest, in comparison to the pepsin concentrations of our experiments.

### Ara h2

In contrast to Ara h1 and Ara h3, Ara h2 was more stable. Even at the high pepsin concentration (10 U of pepsin per μg of substrate), the protein band with the highest molecular weight remained intact for up to 4 minutes. This could only be observed when reducing conditions during the SDS-PAGE analysis were applied. Under non-reducing analysis conditions, virtually no proteolytic breakdown was observed.

The necessity of reduction to visualize proteolysis demonstrates that intra-molecular disulfide bonds keep the hydrolysis products together as a single molecule with a molecular mass similar to the native Ara h2.

Thomas et al. [2004] used 10 U/ug of substrate to investigated the digestibility of Ara h2 as well. In contrast with the present results, they describe a rapid disappearance of both the larger and the smaller isoform of Ara h2. In their discussion, they speculate that a trace of the reducing agent ditrhiotreitiol that was used during the purification of their Ara h2 may have denatured the protein making it more susceptible for digestion by pepsin.

Using a similarly high pepsin concentration (pepsin:protein ratio of 1:2 (w/w)), prior art investigated the digestion of Ara h2 as well. They found a digestion-resistant peptide of 10 kDa, in line with the present observations. The described peptide remained largely intact after subsequent digestion with trypsin/chymotrypsin.

In a recent paper, the digestion of Ara h2 with trypsin and chymotrypsin was described and a peptide of similar size was found. In their analysis, reduction before SDS-PAGE was necessary to visualize hydrolysis, in line with previous results and the present results.

It is likely that pepsin, as well as trypsin/chymotrypsin-induced hydrolysis, results in a similar stable peptide, with minor differences at the N-terminal and/or C-terminal part. This is explained by the fact that proteolysis is restricted by the Ara h2 structure, rather than by the specificity of the applied proteases.

The digestibility characteristics of Ara h2 were described. It was shown that Ara h2 is stable towards pepsin-induced hydrolysis, using a protocol similar to that of Thomas et al. (2004). However, intact Ara h2 migrated on their SDS-PAGE as a single band of approximately 14 kDa, which is not in line with the present understanding of Ara h2. Possibly, the protein was the other abundant 2S albumin, now known as Ara h6.

When studied in more detail, the larger isoform of Ara h2 was more stable than the smaller one. This was also observed others, who digested purified Ara h2 with pepsin. Furthermore, the lowest pepsin:allergen ratio results in one main breakdown product, while at higher ratios at least 2 or 3 distinct bands were visible. Probably, one peptide bond is cleaved relatively easily, while a few other peptide bonds require more rigorous pepsin digestion (e.g. a prolonged time or higher pepsin:allergen ratio).

### Ara h6

Ara h 6 showed a digestion pattern which is very similar to that of Ara h2. More precisely, Ara h6 disappeared with a rate somewhat faster than the larger isoform of Ara h2, and somewhat slower than the smaller isoform of Ara h2. Ara h6 was substantially digested after only 1 minute at the highest pepsin concentration. Lowering the pepsin concentration resulted in a more gradual breakdown, and with the lowest pepsin concentration, some Ara h6 was intact after 30 minutes.

Ara h2 and Ara h6 are both 2S albumins with a high degree of amino acid identity and one could speculate that proteolysis would result in peptides of similar molecular weight.

Digestion of Ara h 6 resulted, as visualized on SDS-PAGE under reducing conditions, in a stable peptide of approximately 10 kDa, similar as for Ara h2, even when the highest pepsin concentration was applied. As for Ara h2, the intra-molecular disulfide bridges of Ara h6 maintain the digestion fragments as a single molecule. Although the digestion of Ara h6 was more rapid than that of (the larger isoform of) Ara h2, a similar large peptide remained for the course of the experiment (1 hour) even when the highest concentration of pepsin is applied.

However, in contrast to Ara h2, digestion of Ara h 6 at the lowest pepsin:allergen ratio resulted in at least 2 breakdown products, and showed (qualitatively) the same band pattern as digestion of Ara h 6 digested with the highest pepsin:allergen ratio.

### Trypsin Digestion of Ara h2

Digestion of both Ara h2 and Ara h6 with pepsin resulted in peptides of approximately 10 kDa, in line with earlier observations. Prior art also found a similar 10 kDa peptide after trypsin digestion of Ara h2. To confirm that observation, trypsin digestion of Ara h2 was done with several concentrations of trypsin (7.2 μg/ml, 24 μg/ml, and 72 μg/ml), with the middle concentration representing the conditions applied.

Indeed, the results were fully consistent with these results. A 9 kDa peptide was described after digestion of Ara h2 with trypsin, and also a 4 kDa peptide was described, but this peptide is hardly visible on their SDS-PAGE analysis.

4 kDa band was not observed in the present experiment with trypsin. Interestingly, two bands were observed in the molecular weight region of 9 and 4 kDa after digestion of Ara h2 and Ara h6 with pepsin. Of course, pepsin has a different specificity to that of trypsin/chymotrypsin, and the results cannot be extrapolated easily.

However, there are many potential cleavage sites for both pepsin and tryspin throughout the sequence of Ara h2 and Ara h6 and only a few of them are cleaved in practice. This observation points in to the direction that the 2S albumin structure may be a more important factor than its primary sequence with regard to susceptibility of peptide bonds in 2S albumins from peanut.

Comparison of Digestion of Ara h1, Ara h2, Ara h3 and Ara h6

The digestibility of allergens can be compared by following the disappearance of the intact protein bands on SDS-PAGE, or by following the existence and subsequent disappearance of peptides that originate from the intact protein bands, both provided that identical experimental conditions are applied.

To our knowledge, this is the first study where the potential gastric digestibility of all of the major peanut allergens is investigated in one set of experiments. One study included both Ara h1 and Ara h2 [Astwood et al., 1996], but as explained in the previous section of the digestibility of Ara h2, it is doubted that the protein they considered as Ara h2 is indeed Ara h2.

On examining the disappearance of the intact allergen bands, it was clear that Ara h1, and both the acidic and basic subunits of Ara h3 were digested rapidly when the conditions suggested by Thomas et al. (2004) were applied.

One could argue that the pepsin:protein ratio is comparatively high in the protocol for Thomas et al. (2004), however, it is accepted that such a ratio may represent stomach conditions [US Pharmacopeia, 1995]. Lowering the pepsin concentration by 10-fold also resulted in a rapid disappearance of both Ara h1 and Ara h3. Even at a 100-fold lower concentration of pepsin, all intact protein bands of these allergens disappeared after less than a minute.

On the other hand, Ara h2, in particular the larger isoform, and to a lesser extent Ara h6, remained intact upon digestion for some time when using the highest pepsin concentration. On lowering the pepsin concentration by 10- and 100-fold, the intact protein bands remained for longer time periods. Where Ara h1 and Ara h3 disappear within 15 seconds (even at the lowest pepsin concentration), Ara h2 and Ara h6 remain for 30-60 minutes, indicating a difference in digestion kinetics of at least 100-fold.

The larger isoform of Ara h2 was most stable of all; it remains intact for several minutes at the highest concentration of pepsin, and for >60 minutes for the lowest concentration of pepsin, indicating that this allergen was digested at least 240-fold more slowly than Ara h1 and Ara h3.

When focusing on peptides that originate from the intact allergens, and the fate of these peptides, it was noticed immediately that the breakdown products of Ara h3 (most abundant at the lowest concentration of pepsin disappeared more quickly than those of the other peanut allergens. Even at this low concentration of pepsin, virtually all breakdown products disappeared after 4 minutes.

For Ara h1, under these conditions, breakdown products remained for the course of the experiment (60 minutes). However, when based on the comparative staining intensities, the breakdown products of the native Ara h1 (after 60 minutes) appear to represent only a fraction of the originally present Ara h1.

In contrast, for Ara h6 and both isoforms of Ara h2, peptides of approximately 10 kDa were generated, obviously more quickly when higher ratios pepsin:allergen were applied. These breakdown products, in their turn, remained for the course of the experiment, even at the highest pepsin:allergen ratio. Comparing the staining intensities of these breakdown products at 60 minutes with that of the native Ara h2 and Ara h6 appears to show that a major fraction of the original allergen is still present as peptides of approximately 10 kDa.

Next to the 10 kDa peptide, a peptide of 4 kDa was described as proteolytic breakdown product of peanut 2S albumin [Lehmann et al., 2006]. Such peptides of <10 kDa were observed for Ara h 2, and such peptides were even more pronounced for Ara h6. Investigating the SDS-PAGE patterns of the peptides that originate from digestion of the different peanut allergens, and their respective stability to further breakdown lead to the following overall picture: both Ara h2 and Ara h6 are degraded to large peptides that remain present during the course of the experiment, while for Ara h3 and to a lesser extent Ara h1, the emerging breakdown products are not stable.

### Digestion Resistant Peptides Found in Ara h2

In order to obtain a high yield of digestion-resistant peptide, the reaction product of the incubation with the highest concentration (at 20 minutes) was taken to investigate the digestion-resistant peptides. Purification of the reaction product showed that multiple peptides in the range of 10 kDa were formed after digestion with trypsin.

The peptides were characterized by N-terminal sequencing and for two peptides, the N-terminus was the same as for the native protein. Earlier work showed peptides with a similar molecular weight but a slightly shifted (3 amino acids) N-terminus indicating proteolytic shortening of the N-terminus in the other studies.

Interestingly, prior art reported that this N-terminal fragment had a mass of about 5 kDa, while the predicted sequence was about 10 kDa, in line with the peptide previously reported. The difference was explained by the removal of amino acids in the C-terminal part of the peptide, but this should then have been extensive.

Of the two N-terminal peptides it was found that the one most abundant had a slightly lower molecular weight than the other. Also found was an abundant peptide of approximately 10 kDa with an N-terminus corresponding to the middle part of Ara h2 (GAGSS), suggesting that the C-terminal part of Ara h2 is digestion resistant as well. This is in agreement with others who also reported a peptide with this N-terminus and with a similar molecular weight. However, others did not identify this peptide.

Sequence data of the peptides found in the present study are aligned with those of earlier reports and shown in FIG. 10. Using a molecular weight range of 9 to 11 kDa to describe the 10 kDa N-terminal peptide found by Sen et al. [2002], the sequence underlined in FIG. 10, panel C, should indicate the cleavage site.

The present data and those from Lehmann et al. [2006] indicated an abundant peptide with the N-terminal sequence GAGS S. To explain this, cleavage should have taken place after the arginine residue preceding GAGSS, giving a molecular weight of 8.6 kDa for the N-terminal peptide. The deviation between 8.6 and 10 kDa can be explained by the imprecision of the analytical method (SDS-PAGE), but Sen et al. [2002], hypothesized that the C-terminus of their N-terminal peptide was extended with about 20 amino acids, leaving no room for a peptide starting with GAGSS.

Disulfide bridge mapping of 2S albumins of peanut allows both options to form a single molecule upon digestion that falls into two parts after reduction. Taken together, the present data are in agreement with earlier work, but indicate a higher degree of heterogeneity of digestion-resistant peptides arising from Ara h2. These peptides have sufficient length to suggest that they could both sensitize susceptible individuals enabling the development of hypersensitivity and subsequently elicit allergic reaction in peanut-allergic individuals.

### Conclusions

By evaluating the potential gastric digestibility of purified major allergens from peanut in one series of experiments, it is concluded that Ara h2 and Ara h6 are far more stable to peptic digestion as compared to Ara h1 and Ara h3.

Digestion-resistant peptides obtained after digestion of Ara h2 with pepsin consist of a pool of relatively large peptides that may be able to elicit allergic reactions. This is not the case for Ara h1 and Ara h3 where peptides that originate from digestion are quickly broken down further. This improved understanding of the comparative gastric stability of the major peanut allergens suggests that immunotherapeutic strategies should be focused on Ara h2 and Ara h6.

### Example 3

Production of Ara h2/Ara h6 Preparations

Peanut acetone powder (150 g, Greer laboratories) was suspended in Tris/HCl buffer (1.5 L, 50 mM) and the suspension was stirred for 1.5 h at room temperature. The suspension was thereafter filtered over a Buchner funnel with a Sefar 07-20/13 filter yielding a solution of 1100 ml. The solution was then filtered through depth filters and subsequently through a 0.2 μm filters yielding the undiluted CPE solution (830 ml). The latter solution was then diluted with Tris/HCl buffer (3160 ml, 50 mM, pH8).

Anion exchange chromatography was used to purify Ara h2 and Ara h6 from the peanut extract. Therefore, a 93 ml Q Sepharose X1 column was equilibrated with Tris/HCl buffer (190 ml, 50 mM, pH 8). The peanut extract was then applied to the column at a flow rate of 20 ml/min. The column was thereafter washed with Tris/HCl buffer (270 ml of 50 mM) and eluted with 50 mM Tris/HCl+200 mM NaCl (700 ml, pH 8). Finally, 640 ml of solution was collected, and concentrated (about 10-fold) over a 10 kD membrane. The final concentrate was filtered over an 0.2 μm filter yielding a 68 ml solution.

To further purify Ara h2 and Ara h6, a size exclusion chromatography was employed. A Superdex 75 column was equilibrated with a 50 mM phosphate buffer, pH 8 with 150 mM NaCl. 23 ml of the concentrated solution was applied to the column at a flow rate of 9 ml/min and fractions of 10 ml were collected. Fractions containing Ara h2 and Ara h6 were pooled and stored at −20° C.

### Modification of Ara h2 and Ara h6 Preparations

### Reduction and Alkylation (RA)

Frozen Ara h2/Ara h6 preparations were thawed through incubation at 30° C. for 30 min and diluted with 100 mM Tris/HCl buffer (pH 8.5) to a final concentration of 1 mg/ml. 1M dithiothreitol (DTT) was added to a final concentration of 5 mM. After 1 hour incubation at 60° C. 0.5M iodoacetamide (IAA) was added to a final concentration of 10 mM and the resulting mixture was incubated for 90 minutes at room temperature in the absence of light.

The reduced-alkylated conglutin was thereafter diafiltered against 50 mM sodium phosphate buffer (pH 8) by using 3 kD centrifuge modules. The preparation was concentrated during the diafiltration (˜0.4 times) and thereafter filtered through a 0.2 nm filter and stored at −20° C.

### Reduction and Alkylation Modification Procedure Followed by Cross-Linking (RAGA)

To the reduced-alkylated Ara h2/Ara h6 preparation 5% glutaraldehyde (GA) was added to a final concentration of 0.4%. After an overnight incubation period at room temperature a 10% glycine solution was added to a final concentration of 0.8%. The reduced, alkylated and cross-linked Ara h2/Ara h6 was diafiltered against 50 mM sodium phosphate buffer (pH 8) by using 3 kD centrifuge modules. After the diafiltration procedure the preparation was concentrated about 4 times. This fraction was filtered through a 0.2 nm filter and stored at −20° C.

### Immunogenicity of the Ara h2 and Ara h6 Preparations

The IgE-binding potencies of the RA and RAGA Ara h2 and Ara h6 preparations were measured as described above. The results are reported in Table 10 below.

TABLE 10
IgE-binding potencies of conglutins after RA and RAGA treatment.

| | Protein Content | |
|---|---|---|
| Sample | (mg/ml) | Relative Potency |
| native | 1.10 | 0.72 |
| RA | 0.83 | 0.00 |
| RAGA | 0.86 | 0.00 |

As can be clearly seen in Table 10, the RA and RAGA modified preparations of Ara h2 and Ara h6 showed a significantly reduced, or even absent, IgE binding as compared to the unmodified preparation.

Additionally, short-term Ara h2 and Ara h6-specific human TCLs were generated and tested for antigen-specificity on day 21. TCLs were tested by stimulation with the indicated preparations (Blank=negative control; native=non-modified Ara h2 and Ara h6 preparation; RA=Ara h2 and Ara h6 preparation modified by reduction and alkylation; RAGA=Ara h2 and Ara h6 preparation modified by reduction, alkylation and glutaraldehyde) at a concentration of 25 μg/ml. A stimulation index (SI) of >2 is considered positive.

The mean SEM of the SI of six TCLs are shown in FIG. 11. From this figure, it can be seen that all Ara h2 and Ara h6 preparations tested are able to induce T cell responses. From this, it is concluded that Ara h2 and Ara h6 preparations modified by reduction and alkylation, or by reduction, alkylation, and glutaraldehyde treatment, do maintain their ability to stimulate specific T cell proliferations.

### Example 4

### Methods

Blood and sera were obtained from patients with peanut allergy, from US-based populations. The average RAST value was RAST class 3, ranging from class 1 to class 5 (specific IgE: 0.35 to >100 kU/L), representing different gradations of peanut allergy [Sampson, 2001].

IgE-Western-blotting was performed as previously described for individual peanut allergen Ara h1, Ara h2, Ara h3, and Ara h6 [Koppelman, 2004] using sera from peanut allergic patients. Basophile degranulation was performed as described earlier for the for individual peanut allergen Ara h1, Ara h2, Ara h3, and Ara h6 [Koppelman, 2004], using blood from the peanut allergic patients from the US-based population.

### Results

The Western-blotting results show that the majority of the patients has IgE directed toward Ara h2 and Ara h6 in their serum. IgE toward Ara h1 was less often found, and IgE towards Ara h3 only in sera of a minor part of the population. The results are summarized in Table 11. It was also observed that the intensity of recognition was much higher for Ara h2 and Ara h6 as compared to Ara h1 and Ara h3.

TABLE 11
Recognition of individual peanut allergens
Ara h1, Ara h2, Ara h3, and Ara h6

| | Fraction of patients | |
|---|---|---|
| | that recognize | Intensity on |
| Allergen | this allergen | Westernblot |
| Ara h 1 | 0.4 | + |
| Ara h 2 | 0.9 | +++ |
| Ara h 3 | 0.2 | +/− |
| Ara h 6 | 0.9 | +++ |

The basophile degranulation results show that basophiles with IgE from peanut allergic patients react in all cases with Ara h2 and Ara h6 at low concentrations of allergen. In contrast, when reactivity was observed with Ara h1 or Ara h3 (not found in all tested cases), this occurred at higher concentrations, indicating a higher potency for Ara h2 and Ara h6 as compared to Ara h1 pr Ara h3 (Table 12).

TABLE 12
Basophile degranulation with individual peanut
allergens Ara h1, Ara h2, Ara h3, and Ara h6

| | Sensitivity of basophiles |
|---|---|
| Allergen | (relative to Ara h1) |
| Ara h1 | 1 (per definition) |
| Ara h2 | 100-fold |
| Ara h3 | 0.5-fold |
| Ara h6 | 100 fold |

### Discussion

For US patients, it has been described that Ara h1 is the most important allergen [Burks, 1991], and comparison with allergen Ara h2 showed for this US based population that Ara h2 was less frequently recognized [Burks, 1992]. For food allergic consumers in the US, Ara h1 is thought to be the most relevant allergens and therefore an analytical method was developed to specifically detect and quantify Ara h1 in food products by two independent (US-based) investigators [Pomes, 2003; Wen, 2005]. No such test have been described for detecting Ara h2 or Ara h6.

It is understood that for the US based population of peanut allergic patients Ara h1 is the most important peanut allergen. Interestingly for a Dutch based population, it was found, in the contrary, that Ara h2 was the most often recognized allergen for peanut allergic patients [Koppelman 2004] and at that Ara h6 was in a similar way as for Ara h2 more often recognized than Ara h1 [Koppelman, 2006; Flinterman, 2007].

Another parameter to judge allergenicity is allergenic potency as can be determined by the potency to release histamine from effector cells like basophiles and mast cells. Such potency comparison was made for the Dutch population (Koppelman, 2003) showing that Ara h2 is up to a hundred fold more potent than Ara h1. However, recent work from a US population showed that a peanut extract that omits Ara h2 is still very allergenic, indicating an important role for other allergens including Ara h1 and Ara h3.

This observation was confused by a recent observation of the same group stating that Ara h2 together with Ara h6 is responsible for the majority of the potency of a peanut extract. Because there is no information on allergen recognition (frequency of recognition of the individual peanut allergens) in US populations, it is unknown which allergens within the peanut are the most important ones. In summary, the observations in peanut allergic patients from a US population are strikingly different compared to those of European peanut allergic patient.

The remarkable differences between the observations in the US populations on the one hand and Dutch population on the other may be explained by racial differences, differences in lifestyle, differences in exposure patters of peanut during childhood and so on.

The relative importance of Ara h1, Ara h3, Ara h2, and Ara h6 were re-evaluated in a US population. Unexpectedly it was observed that also in this population, Ara h2 and h6 are much more frequently recognized than Ara h1 or Ara h3. It was also observed that Ara h2 and Ara h6 are more potent allergens in terms of histamine release as compared to Ara h1 and h3.

These unexpected results may be explained by the fact that in previous studies by Burks either used a population that was not representative, or too small, or by the possible sub-optimal methodology to determine relative importance of the peanut allergens.

The observation that Ara h2 and Ara h6 are more important than Ara h1 and Ara h3 is supported by the results of the digestion experiment (example 2) in which it was shown that Ara h2 and Ara h6 are more resistant to digestion than Ara h1 and Ara h3.

1. Pharmaceutical composition for immunotherapy comprising:
reduced and alkylated naturally occurring Ara h2 and Ara h6, or derivates or isoforms thereof;
wherein said pharmaceutical composition substantially does not comprise Ara h1 and/or Ara h3.

2. Pharmaceutical composition for immunotherapy comprising:
reduced and alkylated naturally occurring Ara h2 or Ara h6, or derivates or isoforms thereof;
wherein said pharmaceutical composition substantially does not comprise Ara h1 and/or Ara h3.

3. Pharmaceutical composition according to claim 1 or claim 2, further comprising
one or more adjuvants and/or pharmaceutically acceptable excipients.

4. Pharmaceutical composition according to claim 1 or 2, wherein said one or more adjuvants comprise aluminium.

5. Pharmaceutical composition according to claim 1 or 2, wherein said Ara h2 and/or Ara h6 are reduced, alkylated and crosslinked.

6. Pharmaceutical composition according to claim 1 or 2, wherein said naturally occurring Ara h2 and/or Ara h6 are derived from peanut.

7. Method for preparing a pharmaceutical composition for immunotherapy comprising:
providing a composition comprising naturally occurring Ara h2 and/or Ara h6, or derivates or isoforms thereof, wherein said composition substantially does not comprise Ara h1 and/or Ara h3;
reducing said composition; and
alkylating the reduced composition.

8. Method according to claim 7, wherein said providing comprises purifying Ara h2 and/or Ara h6.

9. Method according to claim 7, further comprising:
crosslinking said reduced and alkylated composition.

10. Method according to claim 7, further comprising formulating the reduced and alkylated composition with one or more adjuvants and/or pharmaceutically acceptable excipients.

11. Method according to claim 7, wherein said reducing comprising contacting the composition with one or more reducing agents chosen from the group consisting of 2-mercaptoethanol (β-ME), dithiothreitol (DTT), dithioerythritol, cysteine, homocystein, tributylphosphine, sulfite, tris(2-carboxyethyl) phosphine (TCEP), sodium (cyano) borohydride, lye, glutathione, E-mercapto ethylamine, thioglycollic acid, methyl sulfide, and ethyl sulfide.

12. Method according to claim 7, wherein said alkylating comprising contacting the reduced composition with one or more alkylating agents chosen from the group consisting of N-ethylmalimide, cystamine, iodoacetamide, iodoacetic acid, alkylhalogenides; alkylsulfates; alkenes, preferably terminal alkenes (H₂C)═C(H)—R, and enzymes.

13. Method according to claim 9, wherein said crosslinking comprises contacting the reduced and alkylated composition with an aldehyde, preferably glutaraldehyde.

14. Method according to claim 7, wherein said naturally occurring Ara h2 and/or Ara h6 are derived from peanut.

15. A method for immunotherapy treatment comprising administering a pharmaceutical composition according to any of claim 1 to 6 to an allergic patient in need thereof in a pharmaceutically effective dose.

16. A process for modifying an allergen comprising the steps of reduction and treatment with a cross-linking agent.

17. A process according to claim 16, wherein the cross-linking agent is an aldehyde, preferably glutaraldehyde.

18. A process according to claim 16, wherein further comprising alkylation.

19. A process according to claim 16, wherein the treatment with the cross-linking agent is carried out after reduction.

20. A process according to claim 18, wherein the reduction is carried out prior to alkylation.

21. A process according to claim 16, wherein the reduction is carried out using a reducing agent chosen from the group of 2-mercaptoethanol (β-ME), dithiothreitol (DTT), dithioerythritol, cysteine, homocystein, tributylphosphine, sulfite, tris(2-carboxyethyl) phosphine (TCEP), sodium (cyano) borohydride, lye, glutathione, E-mercapto ethylamine, thioglycollic acid, methyl sulfide, ethyl sulfide and combinations thereof.

22. A process according to claim 18, wherein the alkylation is carried out using an alkylating agent chosen from the group of N-ethylmalimide, cystamine, iodoacetamide, iodoacetic acid, alkylhalogenides; alkylsulfates; alkenes, preferably terminal alkenes (H₂C)═C(H)—R, enzymes, and combinations thereof.

23. A process according to claim 16 or 18, wherein the allergen is a protein comprising cystein residues.

24. A process according to claim 16 or 18, wherein the allergen is a recombinant protein or a synthetic peptide.

25. A process according to claim 16 or 18, wherein the allergen is obtained from a vegetable source, preferably a storage protein, from an insect, a mammal or a fish or crustacean.

26. An allergen made by a process according to claim 16 or 18.

27. An allergen according to claim 26 for immunotherapy treatment of an allergy brought about by the native form of said allergen allergen.

28. A pharmaceutical composition comprising an allergen according to claim 26, and a pharmaceutically acceptable carrier and/or an adjuvant.

29. A pharmaceutical composition according to claim 28 having the form of a dosage form chosen from the group of a capsule, tablet, lozenge, dragee, pill, droplets, suppository, aerosol, powder, spray, vaccine, ointment, paste, cream, inhalant, or patch.

30. A method for immunotherapy treatment comprising administering a pharmaceutical composition according to claim 28 or 29 to an allergic patient in need thereof in a pharmaceutically effective dose.


SEQUENCE LISTING (8 sequences)

SEQ ID NO: 1 - protein, 5 aa, Arachis hypogaea
RQQWE

SEQ ID NO: 2 - protein, 14 aa, Arachis hypogaea
ELQGDRRCQSQLER

SEQ ID NO: 3 - protein, 6 aa, Arachis hypogaea
ELQGDF

SEQ ID NO: 4 - protein, 8 aa, Arachis hypogaea
GRQGDSSE

SEQ ID NO: 5 - protein, 5 aa, Arachis hypogaea
GAGSS

SEQ ID NO: 6 - protein, 10 aa, Arachis hypogaea
GAGSSQHQER

SEQ ID NO: 7 - protein, 7 aa, Arachis hypogaea
SQDQQQR

SEQ ID NO: 8 - protein, 172 aa, Arachis hypogaea
MAKLTILVALALFLLAAHASARQQWELQGDRRCQSQLERANLRPCEQHLMQKIQRDEDSYGRDPYSPSQDPYSPSQDPDRRDPYSPSPYDRRGAGSSQHQERCCNELNEFENNQRCMCEALQQIMENQSDRLQGRQQEQQFKRELRNLPQQCGLRAPQRCDLEVESGGRDRY